(19) United States
(12) Patent Application Publication
(10) Pub. No.: US 2009/0011000 A1
(43) Pub. Date: Jan. 8, 2009

(54) Inhibitors of Infection

(76) Inventors: Andreas Hinz, Sassenage (FR); Oliver Lenz, Sint Katelijne Waver (BE); Winfried Weissenhorn, Grenoble (FR)

Correspondence Address: WENDEROTH, LIND & PONACK, L.L.P., 2033 K STREET N. W., SUITE 800, WASHINGTON, DC, 20006-1021, US

(21) Appl. No.: 11/664,682

(22) PCT Filed: Oct. 7, 2005
(86) PCT No.: PCT/IB05/03968

§ 371 Date: Jul. 3, 2007

(30) Foreign Application Priority Data
Oct. 8, 2004 (GB) 0422439.0

(51) Int. Cl. A61K 9/127; C07K 14/00; C12N 15/12; C12N 15/70; C12N 1/00; C12P 21/06; A61K 38/16

(52) U.S. Cl. 424/450; 530/350; 536/23.5; 435/69.1; 435/320.1; 435/243; 514/12

(57) ABSTRACT

Fusion inhibitor peptides are provided comprising a sequence derived from an HR2 domain. In preferred embodiments, the peptides are capable of oligomerization. Also provided are nucleic acids encoding the peptides, vectors comprising the nucleic acids and host cells transformed with the vectors. The peptides may be used as medicaments. Also provided are methods for expressing a protein comprising one or more transmembrane domain(s) in an expression system. The methods comprise fusing a sequence encoding an 18-mer peptide, or a functional equivalent thereof, to a gene encoding the protein and expressing the resultant gene-fusion product in an expression system.

All documents cited herein are incorporated by reference in their entirety.

### TECHNICAL FIELD

This invention is in the field of inhibitors of infection, particularly antivirals. In preferred embodiments, the invention is for preventing human immunodeficiency virus (HIV) infection.

This invention also relates to the field of protein expression. In particular, it relates to the expression of membrane proteins.

### BACKGROUND ART

The entry of enveloped viruses into target host cells requires their respective lipid bilayer membranes to fuse. This cell fusion requires the action of fusion proteins that are embedded in the viral membrane. As an example of this process, the mechanism of HIV-1 entry via the HIV-1 glycoprotein Env has been described in detail.

The HIV-1 envelope glycoprotein gp160 is synthesized as a precursor that is post-translationally cleaved into the receptor binding subunit gp120 and the membrane anchored fusion protein gp41. Trimeric Env gp120/gp41 heterodimers interact sequentially with cellular receptors such as CD4 and chemokine co-receptors CXCR4 or CCR5 in a process that mediates fusion of viral and cellular membranes. Receptor binding induces conformational changes in gp120, which in turn brings about conformational changes in the fusion protein subunit gp41, which ultimately lead to the formation of a six helical bundle core structure. Although the exact mechanism remains to be elucidated, several lines of evidence indicate that receptor binding of gp120 leads to dissociation of gp120 from gp41 and the formation of a gp41 prehairpin, with the fusion peptide targeted to the cellular membrane. Subsequent complete refolding of gp41 into the thermostable six helical bundle structure is thought to pull the two membranes into close proximity and initiate membrane fusion.

The core structure of gp41 is composed of a central triple stranded coiled coil (HR1) with three C-terminal helices (HR2) packing anti-parallel. This gp41 structure is termed a “trimer-of-hairpins”. Structural and computation studies indicate that the trimer-of-hairpins is a conserved motif in the fusion proteins of many enveloped viruses from distant viral families, including Retroviridae, Filoviridae, Paramyxoviridae and Orthomyxoviridae {1}. For example, trimer-of-hairpin structures have been solved for the fusion proteins of numerous viruses, including influenza virus HA2 protein {2}, Ebola virus (EbV) GP2 protein {3}, respiratory syncytial virus (RSV) F1 protein {4}, human T-cell leukemia virus type 1 (HIV-1) p21 protein {5}, mouse hepatitis virus S protein {6} and SARS-coronavirus spike S protein {7}.

The ubiquitous role of fusion proteins in enveloped virus infection makes them promising targets for the development of antiviral agents {1}. For example, efforts have been made to identify agents that can inhibit the action of fusion proteins and therefore block the entry of enveloped viruses into host cells. These agents are commonly referred to as “fusion inhibitors”. Moreover, vaccines based on epitopes in the fusion proteins themselves have also been considered.

### Fusion Inhibitors

Certain agents have been identified that can inhibit the action of fusion proteins and therefore block the entry of enveloped viruses into host cells. One such agent, T-20 (also known as “Enfuvirtide” or “Fuzeon™” {8}), is a prototypic fusion inhibitor anti-HIV drug. It is a linear 36-amino acid synthetic peptide that inhibits the HIV/T-cell interaction by binding to the HR1 heptad-repeat region in gp41 and preventing the conformational changes required for membrane fusion. Enfuvirtide is based on the HR2 sequence (specifically consisting of residues 638 to 673 of the Env protein) and is believed to act as a competitive inhibitor of the natural HR1/HR2 interaction. Similarly, another potent fusion inhibitor based on the HR2 sequence is C34, which consists of residues 628 to 661 of the Env protein. Based on examples such as these, many other fusion inhibitor peptides have been predicted for other viral (and non-viral) fusion events (see, for example reference 9, where analogues of T-20 are identified by 107×178×4, ALLMOTI5 and/or PLZIP search motifs). Consistent with predictions such as these, T-20-like sequences have been shown to inhibit coronavirus viral entry and membrane fusion (see, for example, references 10, 11 and 12). Similarly, a T-20-like sequence derived from paramyxovirus has been shown to inhibit SV5 F protein-mediated cell fusion {13} and a T-20-like sequence derived from the measles virus F1 fusion protein has been shown to inhibit measles virus infectivity {14}.

Accordingly, fusion inhibitors are anticipated for use in the treatment of numerous viral infections other than HIV, including coronavirus, paramyxovirus, influenza virus, Ebola virus, respiratory syncytial virus, human T-cell leukemia virus type 1 and measles virus infections. Moreover, bacterial and other viral infections that may be amenable to treatment with fusion inhibitors are identified in references 9 and 14, and the references cited therein.

Accordingly, there is a need for the provision of further fusion inhibitors that can prevent specific viral and bacterial infections. In particular, there is a need for fusion inhibitors with improved properties compared to known fusion inhibitors. There are a number of disadvantages associated with known fusion inhibitors, such as T-20 {1}. For example, T-20 must be used at high dosages to achieve antiviral activity in vivo. Accordingly, there is a need to provide fusion inhibitors that are more effective at inhibiting membrane fusion. There is also a need to provide fusion inhibitors that have increased bioavailability. Moreover, as T-20 is synthesised artificially, it is very costly to produce. Accordingly, there is a need to develop fusion inhibitors that can be produced at lower cost, for example by recombinant techniques.

Given the polypeptide nature of HR2-derived fusion inhibitors, gene therapy has been seen as an alternative to parenteral administration of large doses of fusion inhibitor peptide. For example, references 15, 16 and 17 disclose genetically engineered treatments for HIV infection by the expression of membrane-anchored gp41 peptides. For this treatment, vectors are used that encode a fusion inhibitor consisting of a peptide derived from gp41 of HIV, e.g. T-20, and a C-terminal transmembrane domain linker via a flexible linker. The C-terminal domain is derived from a heterologous type-1 membrane protein and is selected such that it does not oligomerize.

### Vaccines

The antiviral activity of agents targeting fusion proteins suggests that antibodies directed towards these proteins might also possess neutralising ability. This has been confirmed in HIV-1, where the C-terminal membrane proximal sequence of gp41 contains epitopes for neutralizing antibodies 2F5, 4E10 and Z13 ({18}, {19}, {20}, {21} and {22}). The efficiency of mAb 2F5 in preventing infection and reducing viral load has been demonstrated in a number of studies. Consequently, it is of considerable interest to understand the antigenic properties that are capable of inducing such an immune response. Although the epitopes recognized by 2F5 and 4E10 have been mapped to linear sequences (FIG. 1), peptides or constraint peptides thereof fail to induce 2F5-like immune responses, indicating that additional aspects of the Env protein structure are involved in 2F5-mediated neutralization. Thus, the nature of the antigen that is capable of inducing these immune responses remains elusive {23}.

Accordingly, there is a need to develop effective vaccines based on fusion proteins. In particular, there is a need to develop vaccines that elicit the production of neutralising antibodies in vivo.

The present invention provides one or more of the above needs.

### DISCLOSURE OF THE INVENTION

The present invention is based on the discovery of new and improved fusion inhibitor peptides comprising sequences derived from an HR2 domain, that are preferably capable of forming oligomers, preferably trimers. Moreover, in some embodiments, the peptide shows an improved ability to inhibit fusion compared to known fusion inhibitor peptides. This is particularly the case when the peptide is attached to liposomes via a transmembrane domain.

The present invention is also based on the surprising discovery that the above-described peptide is an effective immunogen. Moreover, in some embodiments, the peptide shows an ability to elicit the production of neutralising antibodies. This is particularly the case when the peptide is attached to liposomes via a transmembrane domain.

Accordingly, in one aspect, the present invention provides a fusion inhibitor peptide comprising a sequence derived from an HR2 domain. Preferably, said fusion inhibitor peptide is capable of oligomerization.

In preferred embodiments, the fusion inhibitor peptide is capable of trimerization.

In some preferred embodiments, the sequence derived from an HR2 domain is linked to a transmembrane domain. In other preferred embodiments, the sequence derived from an HR2 domain is linked to a soluble oligomerization domain. Preferably, the transmembrane domain or soluble oligomerization domain in these embodiments is C-terminal to the sequence derived from an HR2 domain.

### DEFINITIONS

The term “fusion inhibitor”, as used herein, refers to an agent's ability to inhibit or reduce the level of membrane fusion between two or more moieties relative to the level of membrane fusion which occurs between said moieties in the absence of the agent. The moieties may, for example, be cell membranes or viral structures, such as viral envelopes or pili.

In general, the term “HR2 domain”, as used herein, refers to a domain that is a functional equivalent of the HR2 domain of HIV gp41. The HR2 domain of HIV gp41 is the heptad repeat region that immediately precedes, starting from the N-terminus, the transmembrane domain in that protein (see reference 1). For example, the HR2 domain from HIV-1 strain HBR2 gp41 is shown in FIG. 1. Accordingly, the “HR2 domain” of the present invention may be from any viral or bacterial protein in which a domain that is a functional equivalent of the HR2 domain of HIV gp41 has been identified. Various techniques have been developed for identifying domains that are functional equivalents of the HR2 domain of HIV gp41. Such methods are well known to those skilled in the art. For example, the “LearnCoil-VMF” program {24} (available at http://web.wi.mit.edu/kim) can be used to identify suitable domains. Similarly, numerous viral proteins comprising domains that are predicted to be functional equivalents of the HR2 domain of HIV gp41 were identified in reference 9 using the 107×178×4, ALLMOTI5 and/or PLZIP search motifs. These are listed in Tables VI, VII and X-XIV of reference 9. Additionally, numerous non-viral prokaryotic (e.g. bacterial) proteins comprising domains that are predicted to be functional equivalents of the HR2 domain of HIV gp41 were identified using this strategy and are listed in Table VIII of reference 9. Preferred viral proteins comprising domains that are functional equivalents of the gp41 HR2 domain are those proteins belonging to the class I viral fusion protein superfamily {25, 26}. This family includes fusion proteins from Orthomyxoviruses, Paramyxoviruses, Retroviruses, Lentiviruses and Filoviruses. Particularly preferred of the non-gp41 viral proteins comprising domains that are predicted to be functional equivalents of the gp41 HR2 domain are those listed in Table VII of reference 9 (for the avoidance of doubt, reference 9 is incorporated herein in its entirety by reference). Other preferred non-gp41 viral proteins comprising domains that are functional equivalents of the HR2 domain of HIV gp41 are influenza virus HA2 protein, Ebola virus (EbV) GP2 protein, respiratory syncytial virus (RSV) F1 protein, human T-cell leukemia virus type 1 (HTLV-1) p21 protein, mouse hepatitis virus S protein, SARS-coronavirus spike S protein, measles virus F1 protein, the paramxyovirus fusion protein F₁ subunit {13} and the coronavirus spike (S) glycoprotein S2 membrane fusion unit {10}.

In preferred embodiments, the term “HR2 domain” refers to the HR2 domain of HIV gp41.

Examples of sequences derived from HR2 domains that are suitable for the fusion inhibitor peptide of the present invention are described in more detail in the definition of “moiety —B—” below.

The expression “capable of oligomerization”, as used herein, refers to the ability of the fusion inhibitor peptide of the invention to form oligomers in solution or in a lipid bilayer. Said oligomers may be hetero- or homo-oligomers. Preferably, the oligomers are homo-oligomers. In preferred embodiments, the oligomerization is trimerization. In preferred embodiments, the peptide is capable of forming oligomers at room temperature and pressure when at a concentration of 0.1 to 20 (or more) mg/ml in PBS at physiological pH. This property is particularly preferred when the peptide comprises a soluble oligomerization domain, as described below. In other preferred embodiments, the peptide is capable of forming oligomers at room temperature and pressure when at a concentration of 0.1 to 20 (or more) mg/ml in PBS containing 1% non-ionic detergent and at physiological pH. This property is particularly preferred when the peptide comprises a transmembrane domain, for example, that can render the fusion inhibitor peptide capable of oligomerization, as described below. Preferred non-ionic detergents include Triton, NP40, Tween 20, β-OG and zwitterionic detergents such as CHAPS. Particularly preferred detergents are β-OG and CHAPS. In some embodiments, peptides of the invention are capable of oligomerization, preferably trimerization, in a lipid bilayer environment, for example when incorporated into liposomes.

Without wishing to be bound by theory, it is believed that oligomerization, preferably trimerization, results in a multivalent fusion inhibitor peptide oligomer that has multiple binding sites for a target HR1 domain, and is therefore more effective than fusion inhibitor peptides that are not capable of oligomerization. However, it may be that an effect other than oligomerization is responsible for certain of the advantageous properties of the peptide. Accordingly, in some embodiments of the invention, the ability to oligomerize is not necessarily an essential feature.

The term “transmembrane domain”, as used herein, refers to a peptide sequence that is capable of spanning a lipid bilayer. Suitable sequences may be identified from any known membrane-spanning protein. Such proteins are well known to those skilled in the art. Moreover, there are numerous methodologies that are known to those skilled in the art for predicting whether a sequence may serve as a transmembrane domain (see, for example, references 27, 28, 29, 30, 31 and 32). Particularly preferred transmembrane domains for the fusion inhibitor peptide of the present invention are transmembrane domains that can render the fusion inhibitor peptide capable of oligomerization, as defined above. Suitable sequences may be derived from any known membrane-spanning protein that assembles into oligomers, preferably trimers, under suitable conditions (e.g. proteins belonging to the class I viral fusion protein superfamily, such as gp41, the influenza virus HA2 protein, Ebola virus (EbV) GP2 protein, respiratory syncytial virus (RSV) F1 protein, human T-cell leukemia virus type 1 (HTLV-1) p21 protein, mouse hepatitis virus S protein, SARS-coronavirus spike S protein, measles virus F1 protein, the paramxyovirus fusion protein F₁ subunit and the coronavirus spike (S) glycoprotein S2 membrane fusion unit; and the MHC Class II-associated invariant chain {33}. (Suitable biochemical tests for identifying whether a given membrane protein forms oligomers, preferably trimers, under a given set of conditions would be well known to those skilled in the art and are exemplified in “Oligomerization and protease sensitivity of gp41ctm” below). In particular, in those embodiments where the HR2 domain of the fusion inhibitor peptide of the present invention is itself derived from an oligomeric, preferably trimeric, membrane-spanning protein (e.g. when it is derived from a member of the class I viral fusion protein superfamily, such as gp41), then the transmembrane domain from that particular membrane-spanning protein is particularly preferred.

The term “soluble oligomerization domain”, as used herein, refers to a peptide sequence that can render the fusion inhibitor peptide capable of oligomerization, as defined above. Suitable domains may be engineered from the transmembrane domains described above by substituting hydrophobic residues positioned on the outside of the oligomeric transmembrane domain assembly with charged residues. Further residues may be changed if necessary to achieve full solubility. Such methods of solubilizing transmembrane domains by protein engineering are known in the art, for example from reference {34}. Other suitable soluble oligomerization domains are soluble helical oligomerization domains. Examples of such domains include triple stranded coiled coil regions such as a trimeric version of the 30 amino acid coiled coil in GCN4. A suitable version is described in reference 35, wherein the “a” and “d” heptad repeat positions have been replaced with isoleucine. Such an amino acid sequence (see SEQ ID NO: 7) has been used for trimerization/stabilization of gp41 in references 36 and 37. The sequence of SEQ ID NO: 7, or a functional equivalent thereof, is preferred as a soluble oligomerization domain for the present invention. Other examples of suitable domains include parallel triple stranded coiled coil sequences, preferably human, which have the same helical register as the trimeric version of the coiled coil in GCN4 referred to above. Preferably, the coiled coil sequence is between 20 and 35, more preferably 23 and 30 amino acid residues long. More preferably, it is as long as the HIV gp41 transmembrane domain (23 residues).

The term “antiviral”, as used herein, refers to the peptide's ability to inhibit viral infection of cells, via, for example, cell-cell fusion or free virus infection. Such infection may involve membrane fusion, as occurs in the case of enveloped viruses, or some other fusion event involving a viral structure and a cellular structure.

The term “antibacterial”, as used herein, refers to the peptide's ability to inhibit bacterial infection of cells, via, for example, cell-cell fusion or free bacterial infection. Such infections may involve membrane fusion or some other fusion event involving a bacterial structure and a cellular structure.

### Peptides

The peptide of the invention will typically be a polypeptide e.g. consisting of between 2 and 500 amino acids. The polypeptide preferably consists of no more than 200 amino acids (e.g. no more than 190, 180, 170, 160, 150, 140, 130, 120, 110, 100, 90, 80, 70, 60 or no more than 50).

The peptide may have the formula NH₂-A-B—C-D-E-COOH, wherein: -A- is an optional N-terminus amino acid sequence consisting of a amino acids; —B— is a sequence derived from an HR2 domain; —C— is an optional amino acid sequence consisting of c amino acids; -D- is a transmembrane domain or a soluble oligomerization domain and -E- is an optional C-terminus amino acid sequence consisting of e amino acids. In this embodiment, it is preferred that the peptide is capable of oligomerization.

As defined above, -A- is an optional N-terminus amino acid sequence consisting of a amino acids. The value of a is generally at least 1 (e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 350, 400, 450, 500, etc.), but can be zero (i.e. -A- is absent). Examples of typical -A- moieties include leader sequences to direct protein trafficking, or short peptide sequences which facilitate cloning or purification (e.g. histidine tags i.e. Hisₙ where n=3, 4, 5, 6, 7, 8, 9, 10 or more; and protease sites, e.g. TEV protease sites, Enterokinase protease sites etc.). In a preferred embodiment, the sequence of moiety -A- is SEQ ID NO: 20, or a functional equivalent thereof. In some embodiments, moiety -A- is, or terminates at its N-terminus with, a methionine residue. Other suitable N-terminus amino acid sequences will be apparent to those skilled in the art.

As defined above, —B— is a sequence derived from an HR2 domain. The function of moiety —B— is to inhibit the interaction of an HR1 domain with an HR2 domain. The term “HR1 domain”, within this context, refers to a domain that is a functional equivalent of the HR1 domain of HIV gp41. The HR1 domain of HIV gp41 is the heptad repeat region that follows, starting from the N-terminus, the fusion peptide in that protein (see reference 1). For example, the HR1 domain from HIV-1 strain HXBR2 gp41 is shown in FIG. 1. Like the HR2 domain, the HR1 domain of the present invention may be from any viral or bacterial protein in which a domain that is a functional equivalent of the HR1 domain of HIV gp41 has been identified. Strategies for identifying such proteins are described above. Exemplary sequences for moiety —B— that are considered part of the present invention are known in the art. For example, numerous sequences from a variety of viral and bacterial sources are given in Table V of reference 14 and Tables VI-VIII and X-XIV of reference 9. Preferably, the function of moiety —B— is to inhibit the interaction of an HR1 domain with an HR2 domain, wherein both domains are found in the same protein. Exemplary sequences for this embodiment are disclosed in references 10, 11, 12, 13 and 14, and in a preferred embodiment, the sequence is the C8 peptide (derived from FIV Env residues 770 to 777 {38}), or a functional equivalent thereof. In particularly preferred embodiments, the function of moiety —B— is to inhibit the interaction of gp41 HR1 with gp41 HR2. Accordingly, in a preferred embodiment, the sequence of moiety —B— is derived from the HR2 domain of gp41. Exemplary sequences derived from the HR2 domain of gp41 that are considered part of the present invention are again known in the art (see, for example, reference 1). Exemplary sequences include T-20 (derived from Env residues 638 to 673 {39}), C34 (derived from Env residues 628 to 661 {40}) and T-1249 ({41}). In a preferred embodiment, the sequence of moiety —B— is the same as the sequence of T-20, C34, or T-1249, or a functional equivalent thereof. In a particularly preferred embodiment, the sequence is SEQ ID NO: 2, or a functional equivalent thereof.

In some embodiments, the amino acid sequence of -A- shares less than x % sequence identity to the a amino acids which are N-terminal of sequence —B— in the specific protein from which —B— is derived. For example, when said protein is gp41 from HIV-1 strain HXBR2, the a amino acids are as given in SEQ ID NO: 3. In general, the value of x is 60 or less (e.g. 50, 40, 30, 20, 10 or less). In preferred embodiments, the sequence of -A- is not SEQ ID NO: 3. In other preferred embodiments, -A- is absent altogether.

As defined above, —C— is an optional amino acid sequence consisting of c amino acids. The value of c is generally at least 1 (e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 350, 400, 450, 500, etc.), but can be zero (i.e. —C— is absent).

In some embodiments, the amino acid sequence of —C— shares at least y % sequence identity to the c amino acids which are C-terminal to sequence —B— in the specific protein from which —B— is derived but N-terminal to the transmembrane domain in said protein sequence. For example, when said protein is gp41 from HIV-1 strain HXBR2, the c amino acids are as given in SEQ ID NO: 4. In another example, when said protein is gp41 from HIV-1 strains MN, 89.6 or JR-FL, the c amino acids are as given in SEQ ID NO: 5. In preferred embodiments, the sequence of —C— shares at least y % sequence identity across its length to SEQ ID NO: 4. In other preferred embodiments, the sequence of —C— shares at least y % sequence identity across its length to SEQ ID NO: 5. In general, the value of y is 50 or more (e.g. 60%, 70%, 80%, 85%, 90%, 95%, 96%, 97%, 98% or 99% or more).

As defined above, -D- is a transmembrane domain or a soluble oligomerization domain. The function of the transmembrane domain or soluble oligomerization domain in the peptides of the invention and suitable examples of such domains are discussed in the definitions of these terms given above. In preferred embodiments, the sequence of -D- is SEQ ID NO: 6, or a functional equivalent thereof. A suitable functional equivalent of SEQ ID NO: 6 is given in SEQ ID NO: 21. In other preferred embodiments, the sequence of -D- is SEQ ID NO: 7, or a functional equivalent thereof.

As defined above, -E- is an optional C-terminus amino acid sequence consisting of e amino acids. The value of e is generally at least 1 (e.g. at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 350, 400, 450, 500, etc.), but can be zero (i.e. -E- is absent). Examples of typical -E- moieties include sequences to direct protein trafficking, short peptide sequences which facilitate cloning or purification (e.g. comprising histidine tags i.e. Hisₙ where n=3, 4, 5, 6, 7, 8, 9, 10 or more), or sequences which enhance protein stability. Other suitable C-terminus amino acid sequences will be apparent to those skilled in the art. In certain embodiments, the function of -E- is to facilitate expression of the protein in an expression system, preferably a prokaryotic expression system. In such embodiments, the amino acid sequence of -E- is preferably SEQ ID NO: 8, or a functional equivalent thereof.

In some embodiments, the amino acid sequence of -E- shares less than z % sequence identity to the e amino acids which are C-terminal to the transmembrane domain in the specific protein from which —B— is derived. For example, when said protein is gp41 from HIV-1 strain HXBR2, the e amino acids are as given in SEQ ID NO: 9. In general, the value of z is 60 or less (e.g. 50, 40, 30, 20, 10 or less). In preferred embodiments, the sequence of -E- is not SEQ ID NO: 9.

In other embodiments, the amino acid sequence of -E- shares at least z₁% sequence identity to f amino acids of the e amino acids which are C-terminal to the transmembrane domain in the specific protein from which —B— is derived. For example, when said protein is gp41 from HIV-1 strain HXBR2, the e amino acids are as given in SEQ ID NO: 9. In general, the value of f is 90 or less (e.g. 80, 70, 60, 50, 40, 30, 20, 10 or less). In preferred embodiments, the value of f is 10 or less (e.g. 9, 8, 7, 6, 5, 4, 3, 2 or 1). In a particular embodiment, when said protein is gp41 from HIV-1 strain HXBR2, the value of f may be 5. In general, the value of z₁ is 50 or more (e.g. 60%, 70%, 80%, 85%, 90%, 95%, 96%, 97%, 98% or 99% or more). In a preferred example of this embodiment, the sequence of -E- is SEQ ID NO: 22, or a functional equivalent thereof.

In some preferred embodiments, -E- is absent altogether.

The value of a+e may be 0 or greater (e.g. at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 350, 400, 450, 500 etc.). It is preferred that the value of a+e is at most 1000 (e.g. at most 900, 800, 700, 600, 500, 450, 400, 350, 300, 250, 200, 190, 180, 170, 160, 150, 140, 130, 120, 110, 100, 90, 80, 70, 60, 50, 40, 30, 25, 20, 19, 18, 17, 16, 15, 14, 13, 12, 11, 10, 9, 8, 7, 6, 5, 4, 3, 2).

The value of a+f may be 0 or greater (e.g. at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 350, 400, 450, 500 etc.). It is preferred that the value of a+f is at most 1000 (e.g. at most 900, 800, 700, 600, 500, 450, 400, 350, 300, 250, 200, 190, 180, 170, 160, 150, 140, 130, 120, 110, 100, 90, 80, 70, 60, 50, 40, 30, 25, 20, 19, 18, 17, 16, 15, 14, 13, 12, 11, 10, 9, 8, 7, 6, 5, 4, 3, 2).

Preferably, component —B—C— of the above-noted formula comprises the monoclonal antibody 2F5 core or extended epitope (SEQ ID NOS: 10 or 11), or a functional equivalent thereof. Preferably, component —B—C— comprises the monoclonal antibody ZI3 epitope (SEQ ID NO: 12), or a functional equivalent thereof. Preferably, component —B—C— comprises the monoclonal antibody 4E10 epitope (SEQ ID NO: 13), or a functional equivalent thereof. More preferably, component —B—C— comprises any two or three of these epitopes, or functional equivalents thereof.

In some polypeptides, the amino acid sequences of the -A-, —B—, —C—, -D- and -E- moieties may contain m amino acid substitutions, where m is an integer. The in amino acids are typically substituted by A, C, D, E, F, G, H, I, K, L, M, N, P, Q, R, S, T, V, W, or Y. Each of the m substitutions may be the same or different as the others. The substitution is preferably by G or, more preferably, by A. The substituting amino acid may be an L- or a D-amino acid but, where the other amino acids all share a single stereo-configuration (i.e. all D- or all L-), the substituting amino acid preferably also has that stereo-configuration (although, of course, G has no stereoisomers).

The invention also provides a peptide, comprising amino acid sequence -A-B—C-D-E-, wherein: -A- is an optional methionine residue; —B— is an amino acid sequence with at least a % sequence identity to SEQ ID NO: 2; —C— is an amino acid sequence with at least b % sequence identity to SEQ ID NO: 4 or SEQ ID NO: 5; -D- is an amino acid sequence with at least c % sequence identity to SEQ ID NO: 6 or SEQ ID NO: 7; -E- is an optional amino acid sequence with at least d % sequence identity to SEQ ID NO: 8. In this embodiment, it is preferred that the peptide is capable of oligomerization. However, in some embodiments, being capable of oligomerization is not an essential feature of the peptide.

The value of a is 50 or more. The value of b is 50 or more. The value of c is 50 or more. The value of d is 50 or more. The values of a, b, c and d are independent of each other, and typical values are 60, 70, 80, 90, 95, 96, 97, 98, 99 or 100. Preferably, the value of d is 100.

Preferably, the peptide comprises SEQ ID NOS: 14, 15, 16 or 17, or a functional equivalent thereof. More preferably, the peptide consists of SEQ ID NOS: 14, 15, 16 or 17, or a functional equivalent thereof.

The invention also provides a peptide, comprising amino acid sequence -A-B—C-D-E-, wherein: -A- is an optional amino acid sequence with at least a₁% sequence identity to SEQ ID NO: 20; —B— is an amino acid sequence with at least b,% sequence identity to SEQ ID NO: 2; —C— is an amino acid sequence with at least c₁% sequence identity to SEQ ID NO: 4 or SEQ ID NO: 5; -D- is an amino acid sequence with at least d₁% sequence identity to SEQ ID NO: 21 or SEQ ID NO: 7; -E- is an optional amino acid sequence with at least e₁% sequence identity to SEQ ID NO: 22. In this embodiment, it is preferred that the peptide is capable of oligomerization. However, in some embodiments, being capable of oligomerization is not an essential feature of the peptide. In preferred embodiments, at least one of -A- and -E- are present in the peptide. More preferably, both -A- and -E- are present in the peptide.

The value of a₁ is 50 or more. The value of b₁ is 50 or more. The value of c₁ is 50 or more. The value of d₁ is 50 or more. The value of e₁ is 50 or more. The values of a₁, b₁, c₁, d₁ and e₁ are independent of each other, and typical values are 60, 70, 80, 90, 95, 96, 97, 98, 99 or 100.

Preferably, the peptide comprises SEQ ID NOS: 23, 24, 25, 26, 27 or 28, or a functional equivalent thereof. More preferably, the peptide consists of SEQ ID NOS: 23, 24, 25, 26, 27 or 28, or a functional equivalent thereof.

The present invention also provides truncations of the peptides of the invention. For example, the N-terminus may be truncated by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 350, 400, 450, 500 etc. amino acid residues. Such truncated versions of the peptides of the present invention may be useful as fusion inhibitors and/or as immunogens. In some embodiments, the truncation comprises the deletion of a methionine residue at the N-terminus of the peptide.

In preferred embodiments, the peptide comprises an N-terminally truncated form of SEQ ID NOS: 14, 15, 16 or 17, or a functional equivalent thereof, wherein said truncated form specifically binds one or more of the monoclonal antibodies 2F5, 4E10 and Z13. Accordingly, the present invention provides peptides comprising SEQ ID NOS: 14, 15, 16 or 17, or a functional equivalent thereof, wherein the N-terminus has been truncated by up to and including 42 amino acid residues (wherein the 4E10 epitope forms the new N-terminus of the peptide). For example, the N-terminus may be truncated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or 42 amino acid residues. Particularly preferred truncations include truncations of 42, 36, 33, 31, 27, 25 and 1 amino acid(s) from the N-terminus. In another preferred embodiment, the peptide comprises SEQ ID NO: 18, or a functional equivalent thereof.

In other preferred embodiments, the peptide comprises an N-terminally truncated form of SEQ ID NOS: 23, 24, 25, 26, 27 or 28, or a functional equivalent thereof, wherein said truncated form specifically binds one or more of the monoclonal antibodies 2F5, 4E10 and Z13. For example, the peptide comprises an N-terminally truncated form of SEQ ID NOS: 27 or 28, or a functional equivalent thereof, wherein said truncated form specifically binds one or more of the monoclonal antibodies 2F5, 4E10 and Z13. Accordingly, the present invention provides peptides comprising SEQ ID NOS: 27 or 28, or a functional equivalent thereof, wherein the N-terminus has been truncated by up to and including 42 amino acid residues (wherein the 4E10 epitope forms the new N-terminus of the peptide). For example, the N-terminus may be truncated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41 or 42 amino acid residues. Particularly preferred truncations include truncations of 42, 36, 33, 31, 27, 25 and 1 amino acid(s) from the N-terminus.

The present invention also provides elongated forms of the (optionally truncated) peptides of the present invention. For example, the N-terminus may be elongated by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 150, 200, 250, 300, 350, 400, 450, 500 etc. amino acid residues. Such elongated versions of the peptides of the present invention may also be useful as fusion inhibitors and/or as immunogens. The additional N-terminus sequence in a peptide of this embodiment may be, or terminate at its N-terminus with, a methionine residue. The additional N-terminus sequence may comprise N-terminal leader sequences to direct protein trafficking, or short peptide sequences which facilitate cloning or purification (e.g. histidine tags i.e. Hisₙ where n=3, 4, 5, 6, 7, 8, 9, 10 or more). The additional N-terminus sequence may comprise a sequence that is designed to enhance the presentation of epitopes that are C-terminal to the additional N-terminus sequence. Suitable sequences for enhancing the presentation of epitopes that are C-terminal to the additional N-terminus sequence may include sequences that are N-terminal to the epitopes in the native protein from which they are derived. Other suitable sequences may include sequences that stabilise oligomerization (preferably trimerization), such as the soluble oligomerization domains defined above (e.g. SEQ ID NO: 7, or a functional equivalent thereof). For example, in preferred embodiments, the additional N-terminus sequence comprises a sequence that enhances the binding of one or more of the monoclonal antibodies 2F5, 4E10 and Z13. In such embodiments, it is preferred that the additional N-terminus sequence comprises sequence that is N-terminal to these epitopes in native gp41, or a soluble oligomerization domain as defined above (e.g. SEQ ID NO: 7, or a functional equivalent thereof). An example of an N-terminally elongated form of a peptide of the invention, which comprises an N-terminus methionine residue, a short N-terminal peptide sequence to facilitate purification and an N-terminal soluble oligomerization domain is presented in SEQ ID NO: 19.

In preferred embodiments, peptides of the invention can inhibit the interaction of HR1 domains with HR2 domains.

Accordingly, in some embodiments, peptides of the invention are capable of inhibiting cell fusion in a suitable cell fusion assay. Assays for cell fusion events are well known to those of skill in the art. Cell fusion assays are generally performed in vitro. Such an assay may comprise culturing cells which, in the absence of any treatment, would undergo an observable level of syncytial formation. For example, uninfected cells may be incubated in the presence of cells chronically infected with a virus (e.g. HIV) that induces cell fusion. For the assay, cells are incubated in the presence of a peptide to be assayed. For each peptide, a range of peptide concentrations may be tested. This range should include a control culture wherein no peptide has been added. Standard conditions for culturing cells, well known to those skilled in the art, are used. After incubation for an appropriate period (24 hours at 37° C., for example) the culture is examined microscopically for the presence of multinucleated giant cells, which are indicative of cell fusion and syncytial formation Well known stains, such as crystal violet stain, may be used to facilitate the visualization of syncytial formation.

In preferred embodiments, peptides of the invention are capable of inhibiting viral infection. Assays for measuring antiviral activity are well known to those of skill in the art. For example, the effect of peptides of the invention on total viral DNA or viral RNA or protein production (measured by western blot or ELISA, for example) may be tested. Similarly, the effect of peptides of the invention on syncytia formation or infection by cell-free virus may be tested. Using appropriate assays, the relative antiviral activity of the peptides against a given strain of virus and/or the strain specific inhibitory activity of the peptide may be determined.

More preferably, peptides of the invention can inhibit the interaction of gp41 HR1 with gp41 HR2. Accordingly, in some embodiments, peptides of the invention are capable of inhibiting cell fusion in a suitable gp41-mediated cell fusion assay. Suitable assays for testing whether a fusion inhibitor peptide is capable of inhibiting gp41-mediated cell fusion are well known in the art. For example, peptides of the invention are preferably able to inhibit gp41-mediated cell fusion as measured by the “Cell Fusion Assay” of reference 41. In other embodiments, peptides of the invention are capable of inhibiting HIV infection. Suitable assays for testing whether a fusion inhibitor peptide is capable of inhibiting infection are well known in the art. For example, peptides of the invention are preferably able to inhibit HIV infection as measured by the “Magi CCR-5 Infectivity Assay”, “Reverse Transcriptase Assay”, “Human PBMC Infectivity/Neutralization Assay” or “In Vivo HU-PBMC SCID Model of HIV-1 Infection” of reference 41, or the “HIV Infectivity Assay” of reference 42, or any other suitable assay known in the art.

Preferably, peptides of the invention are able to inhibit HIV infection as measured by the single round infection assay described in reference 43. A suitable version of this assay is described in detail in “HIV Neutralization assay” below.

Peptides of the invention (including oligopeptides and polypeptides, collectively “peptides”) may be linear, branched or cyclic, but they are preferably linear chains of amino acids. Where cysteine residues are present, peptides of the invention may be linked to other peptides via disulfide bridges. Peptides of the invention may comprise L-amino acids and/or D-amino acids. The inclusion of D-amino acids may be preferred in order to confer resistance to mammalian proteases.

The N-terminus residue of a peptide of the invention may be covalently modified. Suitable covalent groups include, but are not limited to: acetyl (as in Fuzeon™); a hydrophobic group; carbobenzoxyl; dansyl; T-butyloxycarbonyl; amido; 9-fluorenylmethoxy-carbonyl (FMOC); a lipid; a fatty acid; polyethylene; carbohydrate; etc.

Similarly, the C-terminus residue of a peptide may be covalently modified (e.g. carboxamide, as in Fuzeon™, etc.). Suitable covalent groups include, but are not limited to: acetyl; a hydrophobic group; amido; carbobenzoxyl; dansyl; T-butyloxycarbonyl; 9-fluorenylmethoxy-carbonyl (FMOC); a lipid; a fatty acid; polyethylene; carbohydrate; etc.

### Processes

Peptides of the invention may be produced by various means.

A preferred method for production is biological synthesis, e.g. the peptides may be produced by translation. This may be carried out in vitro or in vivo. Biological methods are in general restricted to the production of peptides based on L-amino acids, but manipulation of translation machinery (e.g. of aminoacyl-tRNA molecules) can be used to allow the introduction of D-amino acids (or of other non-natural amino acids, such as iodotyrosine or methylphenylalanine, azidohomoalanine, etc.) {44}.

Production of peptides by biological means gives peptides with an N-terminus methionine residue. Where the N-terminus of a peptide of the invention is not a methionine then this residue (and any other extraneous residues) will have to be removed e.g. by proteolytic digestion.

To facilitate biological synthesis of peptides, the invention provides nucleic acid that encodes a peptide of the invention. The nucleic acid may be DNA or RNA (or hybrids thereof), or their analogues, such as those containing modified backbones (e.g. phosphorothioates) or peptide nucleic acids (PNA). It may be single-stranded (e.g. mRNA) or double-stranded, and the invention includes both individual strands of a double-stranded nucleic acid (e.g. for antisense, priming or probing purposes). It may be linear or circular. It may be labelled. It may be attached to a solid support.

Nucleic acid according to the invention can, of course, be prepared in many ways e.g. by chemical synthesis (e.g. phosphoramidite synthesis of DNA) in whole or in part, by nuclease digestion of longer molecules, by ligation of shorter molecules, from genomic or cDNA libraries, by use of polymerases etc.

Accordingly, the present invention also provides vectors (e.g. plasmids) comprising nucleic acid of the invention (e.g. expression vectors and cloning vectors) and host cells (prokaryotic or eukaryotic) transformed with such vectors.

The invention also provides a process for producing a peptide of the invention, comprising the step of culturing a host cell transformed with nucleic acid of the invention under conditions that induce expression of the peptide.

Suitable expression systems for use in the present invention are well known to those of skill in the art and many are described in detail in references 45 and 46.

Generally, any system or vector that is suitable to maintain, propagate or express nucleic acid molecules to produce a peptide in the required host may be used. The appropriate nucleotide sequence may be inserted into an expression system by any of a variety of well-known and routine techniques, such as, for example, those described in reference 45. Generally, the encoding gene can be placed under the control of a control element such as a promoter, ribosome binding site (for bacterial expression) and, optionally, an operator, so that the DNA sequence encoding the desired peptide is transcribed into RNA in the transformed host cell.

Examples of suitable expression systems include, for example, chromosomal, episomal and virus-derived systems, including, for example, vectors derived from: bacterial plasmids, bacteriophage, transposons, yeast episomes, insertion elements, yeast chromosomal elements, viruses such as baculoviruses, papova viruses such as SV40, vaccinia viruses, adenoviruses, fowl pox viruses, pseudorabies viruses and retroviruses, or combinations thereof, such as those derived from plasmid and bacteriophage genetic elements, including cosmids and phagemids. Human artificial chromosomes (HACs) may also be employed to deliver larger fragments of DNA than can be contained and expressed in a plasmid.

Particularly suitable expression systems include microorganisms such as bacteria transformed with recombinant bacteriophage, plasmid or cosmid DNA expression vectors; yeast transformed with yeast expression vectors; insect cell systems infected with virus expression vectors (for example, baculovirus); plant cell systems transformed with virus expression vectors (for example, cauliflower mosaic virus, CaMV; tobacco mosaic virus, TMV) or with bacterial expression vectors (for example, Ti or pBR322 plasmids); or animal cell systems. Cell-free translation systems can also be employed to produce the peptides of the invention.

For long-term, high-yield production of a recombinant peptide, stable expression is preferred. For example, cell lines that stably express the peptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Mammalian cell lines available as hosts for expression are known in the art and include many immortalised cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Bowes melanoma and human hepatocellular carcinoma (for example Hep G2) cells and a number of other cell lines.

In the baculovirus system, the materials for baculovirus/insect cell expression systems are commercially available in kit form from, inter alia, Invitrogen, San Diego Calif. (the “MaxBac” kit). These techniques are generally known to those skilled in the art and are described fully in reference 47. Particularly suitable host cells for use in this system include insect cells such as Drosophila S2 and Spodoptera Sf9 cells.

There are many plant cell culture and whole plant genetic expression systems known in the art. Examples of suitable plant cellular genetic expression systems include those described in references 48, 49, 50 and 51. In particular, all plants from which protoplasts can be isolated and cultured to give whole regenerated plants can be utilised, so that whole plants are recovered which contain the transferred gene. Practically all plants can be regenerated from cultured cells or tissues, including but not limited to all major species of sugar cane, sugar beet, cotton, fruit and other trees, legumes and vegetables.

Examples of particularly preferred prokaryotic expression systems include those that use streptococci, staphylococci, E. coli, Streptomyces and Bacillus subtilis as host cells.

Examples of particularly suitable fungal expression systems include those that use yeast (for example, S. cerevisiae) and Aspergillus as host cells.

An alternative to biological synthesis for producing peptides of the invention involves in vitro chemical synthesis {52, 53}. Solid-phase peptide synthesis is particularly preferred, such as methods based on t-Boc or Fmoc {54} chemistry. Enzymatic synthesis {55} may also be used in part or in full. Where D-amino acids are included in peptides of the invention it is preferred to use chemical synthesis.

Accordingly, the invention also provides a process for producing a peptide of the invention, comprising the step of synthesising the peptide by chemical means. The peptide may be synthesised in whole or in part by such chemical means.

### Drug Design and Peptidomimetics

Peptides of the invention are useful antibacterials or antivirals in their own right. However, they may be refined to improve antibacterial or antiviral activity or to improve pharmacologically important features such as bioavailability, toxicology, metabolism, pharmacokinetics, etc. The peptides may therefore be used as lead compounds for further research and refinement.

Peptides of the invention can be used for designing peptidomimetic molecules {e.g. references 56 to 62} with antibacterial or antiviral activity. These will typically be isosteric with respect to the peptides of the invention but will lack one or more of their peptide bonds. For example, the peptide backbone may be replaced by a non-peptide backbone while retaining important amino acid side chains.

Accordingly, the present invention also provides a peptidomimetic compound of a peptide the invention, wherein the peptidomimetic compound is a fusion inhibitor.

The peptidomimetic molecule may comprise sugar amino acids {63}. Peptoids may also be used.

### Pharmaceutical Compositions

The invention provides a pharmaceutical composition comprising (a) a peptide of the invention and (b) a pharmaceutical carrier.

Component (a) is the active ingredient in the composition, and this is present at a therapeutically effective amount e.g. an amount sufficient to inhibit infection. The precise effective amount for a given patient will depend upon their size and health, the nature and extent of infection, and the composition or combination of compositions selected for administration. The effective amount can be determined by routine experimentation and is within the judgement of the clinician. For purposes of the present invention, an effective dose will generally be from about 0.01 mg/kg to about 5 mg/kg, or about 0.01 mg/kg to about 50 mg/kg or about 0.05 mg/kg to about 10 mg/kg. Pharmaceutical compositions based on peptides are well known in the art (e.g. FUZEON™). Peptides may be included in the composition in the form of salts and/or esters.

Carrier (b) can be any substance that does not itself induce the production of antibodies harmful to the patient receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers can include liquids such as water, saline, glycerol and ethanol. Auxiliary substances, such as wetting or emulsifying agents, pH buffering substances, and the like, can also be present in such vehicles. Liposomes are suitable carriers. A thorough discussion of pharmaceutical carriers is available in reference 64.

In preferred embodiments, the carriers are liposomes. “Liposome” refers to a generally spherical cluster or aggregate of amphipathic compounds, including lipid compounds, typically in the form of one or more concentric layers, for example, monolayers and/or bilayers. The liposomes may be formulated, for example, from ionic lipids and/or non-ionic lipids. The preparation of suitable liposomes would be well known to those of skill in the art (see, for example, reference 65). The peptide may be incorporated in the liposome in a variety of ways. Generally speaking, the peptide may be incorporated by being associated covalently or non-covalently with one or more of the materials which are included in the liposomes. In a preferred embodiment, the peptide is incorporated in the liposome via non-covalent associations. As known to those skilled in the art, non-covalent association is generally a function of a variety of factors, including, for example, the polarity of the involved molecules and the charge (positive or negative), if any, of the involved molecules, and the like. Non-covalent bonds are preferably selected from the group consisting of ionic interaction, dipole-dipole interaction, hydrogen bonds, hydrophilic interactions, van der Waal's forces, and any combinations thereof. Preferably, the peptide is incorporated in the liposome by means of a transmembrane domain that forms part of the peptide. Preferably, the peptide is incorporated in the liposome such that sequence derived from an HR2 domain is on the outside face of the liposome.

Pharmaceutical compositions of the invention may be prepared in various forms. For example, the compositions may be prepared as injectables, either as liquid solutions or suspensions. Solid forms suitable for solution in, or suspension in, liquid vehicles prior to injection can also be prepared. The composition may be prepared for topical administration e.g. as an ointment, gel, cream or powder. The composition be prepared for oral administration e.g. as a tablet or capsule, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops, as a spray, or as a powder (as described in reference 66). The composition may be lyophilised.

In a particularly preferred embodiment, the peptide of the invention is prepared as a liposomal gel for topical administration, preferably for rectal or vaginal delivery. Suitable liposomal gels for vaginal delivery are known in the art and described in, for example, references 67 and 68.

The pharmaceutical composition is preferably sterile. It is preferably pyrogen-free. It is preferably buffered e.g. at between pH 6 and pH 8, generally around pH 7.

The invention also provides a delivery device containing a pharmaceutical composition of the invention. The device may be, for example, a syringe or an inhaler.

Peptides of the invention may be co-administered with one or more antibacterials or antivirals, preferably those which are active against the bacterium or virus targeted by the peptide of the invention. Compositions of the invention may thus include one or more antibiotics or antiviral agents. For example, it is well known in the art that for HIV treatment to be effective over a long period of time, more than one anti-HIV drug should be administered to the subject. Accordingly, when peptides of the present invention are used in the treatment of HIV infection, combination therapy with other anti-HIV drugs is preferred. In particular, “Highly Active Antiretroviral Therapy” (HAART) is preferred, wherein three or more anti-HIV drugs are taken at the same time. Suitable anti-HIV drugs for use in combination with the peptides of the present invention are known in the art. These include, but are not limited to:
(i) reverse transcriptase inhibitors such as 3TC (“Epivir” or “Lamivudine”), Abacavir (“Ziagen” or “ABC”), AZT (“Retrovir” or “Zidovudine”), Combivir (AZT/3TC combined), Trizivir (AZT/3TC/Abacavir combined) d4T (“Zerit” or “Stavudine”), ddC (“Hivid” or “Zalcitabine”), ddl (“Videx” or “Zalcitabine”) and FTC (“Emtriva” or “Emtricitabine”);
(ii) non-nucleoside reverse transcriptase inhibitors (NNRTIs) such as Delavirdine (“Resciptor”), Efavirenz (“Sustiva”) and Nevirapine (“Viramune”);
(iii) nucleotide reverse transcriptase inhibitors such as Tenofoir (“Viread”);
(iv) protease inhibitors such as Amprenavir (Agenerase), Atazanavir (Reyataz), Indinavir (Crixivan), Lopinavir/Ritonavir (“Kaletra”), Nelfinavir (“Viracept”), Ritonavir (“Norvir”), Saquinavir (“Fortovase” or “Invirase”) and Tipranavir (“PNU-140690”); and
(v) other fusion inhibitors such as T-20 (“Fuzeon” or “Enfuvirtide”).

In some embodiments, the composition is an immunogenic composition (e.g. a vaccine). Vaccines based on peptides are well known in the art.

Immunogenic compositions comprise an immunologically effective amount of peptide antigen, as well as any other of other specified components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials. Dosage treatment may be a single dose schedule or a multiple dose schedule (e.g. including booster doses). The vaccine may be administered in conjunction with other immunoregulatory agents.

The immunogenic composition may include an adjuvant. Exemplary adjuvants are given in reference 69 and include aluminium compounds e.g. aluminium hydroxides (e.g. oxyhydroxides), aluminium phosphates (e.g. hydroxyphosphates, orthophosphates), aluminium sulphates, etc. (e.g. chapters 8 & 9 of reference 70) and other substances that act as immunostimulating agents to enhance the effectiveness of the immunogenic composition (e.g. see Chapter 7 of reference 70). Alum (especially aluminium phosphates and/or hydroxides) is a preferred adjuvant.

Once formulated, the compositions of the invention can be administered directly to the subject. The subjects to be treated can be animals; in particular, human subjects can be treated.

Vaccines according to the invention may either be prophylactic (i.e. to prevent infection) or therapeutic (i.e. to treat disease after infection), but will typically be prophylactic.

As well as peptides of the invention, the immunogenic composition may comprise further antigenic components. For example, the immunogenic composition may also comprise protein antigens. Exemplary antigens are provided in reference 69 and include:
antigens from Helicobacter pylori such as CagA {71 to 74}, VacA {75, 76}, NAP {77, 78, 79}, HopX {e.g. 80}, HopY {e.g. 80} and/or urease.
an antigen from Bordetella pertussis, such as pertussis holotoxin (PT) and filamentous hemagglutinin (FHA) from B. pertussis, optionally also in combination with pertactin and/or agglutinogens 2 and 3 {e.g. references 81 & 82}.
a diphtheria antigen, such as a diphtheria toxoid {e.g. chapter 3 of reference 83} e.g. the CRM₁₉₇ mutant {e.g. 84}.
a tetanus antigen, such as a tetanus toxoid {e.g. chapter 4 of reference 84}.

The immunogenic composition may comprise one or more of these further antigens.

Toxic protein antigens may be detoxified where necessary (e.g. detoxification of pertussis toxin by chemical and/or genetic means {83}).

Where a diphtheria antigen is included in the immunogenic composition it is preferred also to include tetanus antigen and pertussis antigens. Similarly, where a tetanus antigen is included it is preferred also to include diphtheria and pertussis antigens. Similarly, where a pertussis antigen is included it is preferred also to include diphtheria and tetanus antigens.

Antigens are preferably adsorbed to an aluminium salt.

Antigens in the immunogenic composition will typically be present at a concentration of at least 1 μg/ml each. In general, the concentration of any given antigen will be sufficient to elicit an immune response against that antigen.

The vaccine formulations of the invention may be presented in unit-dose or multi-dose containers. For example, sealed ampoules and vials and may be stored in a freeze-dried condition requiring only the addition of the sterile liquid carrier immediately prior to use. The dosage will depend on the specific activity of the vaccine and can be readily determined by routine experimentation.

A number of suitable methods for vaccination and vaccine delivery systems are described in reference 85.

### Medical Treatments and Uses

The invention provides a peptide of the invention for use as a medicament. The invention also provides a method for treating a subject suffering from or at risk of contracting an infection, comprising administering to the subject a pharmaceutical composition of the invention. The invention also provides the use of a pharmaceutical composition of the invention in the manufacture of a medicament for treating a subject.

The invention also provides a method for raising an antibody response in a subject, comprising administering a pharmaceutical composition of the invention to the subject.

The invention also provides a method for immunising a subject, comprising administering a pharmaceutical composition of the invention to the subject.

Infections that may be treated by the pharmaceutical compositions of the present invention include those caused by bacteria and viruses wherein the infection process requires a protein in which a domain that is a functional equivalent of the HR1 domain of HIV gp41 has been identified. Preferred infections are those caused by viruses wherein the infection process requires a protein belonging to the class I viral fusion protein superfamily. Exemplary viruses include orthomyxoviruses, paramyxoviruses, retroviruses, lentiviruses and filoviruses. Particularly preferred viruses include HIV (with HIV-1 being especially preferred), influenza virus, Ebola virus, respiratory syncytial virus, human T-cell leukemia virus, mouse hepatitis virus, SARS-coronavirus and measles virus.

In embodiments wherein a pharmaceutical composition of the invention is used in the treatment of HIV infection, it is preferred that more than one anti-HIV drug is administered to the subject. Accordingly, in the methods and uses of the present invention, it is preferred that the pharmaceutical composition is administered in simultaneous, separate or sequential application with other anti-HIV drugs. In particular, “Highly Active Antiretroviral Therapy” (HAART) is preferred, wherein the peptide is administered in simultaneous, separate or sequential application with three or more anti-HIV drugs. Suitable anti-HIV drugs for use in combination with the pharmaceutical composition of the present invention are known in the art and examples are listed above.

The subject is preferably a mammal, more preferably a human. The human may be an adult or, preferably, a child. A composition intended for children may also be administered to adults e.g. to assess safety, dosage, immunogenicity, etc.

Compositions of the invention will generally be administered directly to a subject. Direct delivery may be accomplished by parenteral injection (e.g. subcutaneously, intraperitoneally, intravenously, intramuscularly, or to the interstitial space of a tissue), or by rectal, oral (e.g. tablet, spray), vaginal, topical, transdermal {e.g. see reference 86} or transcutaneous {e.g. see references 87 & 88}, intranasal {e.g. see reference 89}, ocular, aural, pulmonary or other mucosal administration.

Dosage treatment can be a single dose schedule or a multiple dose schedule.

The uses and methods of the invention can be used therapeutically (e.g. for treating an existing bacterial and viral infections) or prophylactically (e.g. in a situation where contact with infectious agents is expected and where establishment of infection is to be prevented). Therapeutic use is preferred, and efficacy of treatment can be tested by monitoring bacterial or viral titres after administration of the pharmaceutical composition of the invention, or by monitoring symptoms.

### Antibodies

The peptides of the present invention or their immunogenic fragments (comprising at least one antigenic determinant) can be used to generate ligands, such as polyclonal or monoclonal antibodies, that are immunospecific for the polypeptides. Such antibodies may be employed to isolate or to identify clones expressing the polypeptides of the invention or to purify the polypeptides by affinity chromatography. The antibodies may also be employed as diagnostic or therapeutic aids, amongst other applications, as will be apparent to the skilled reader.

The term “immunospecific” means that the antibodies have substantially greater affinity for the polypeptides of the invention than their affinity for other related polypeptides in the prior art. As used herein, the term “antibody” refers to intact molecules as well as to fragments thereof, such as Fab, F(ab′)2 and Fv, which are capable of binding to the antigenic determinant in question. Such antibodies thus bind to the polypeptides of the invention.

By “substantially greater affinity” we mean that there is a measurable increase in the affinity for a polypeptide of the invention as compared with the affinity for related peptides in the prior art.

Preferably, the affinity is at least 1.5-fold, 2-fold, 5-fold 10-fold, 100-fold, 10³-fold, 10⁴-fold, 10⁵-fold or 10⁶-fold greater for a polypeptide of the invention than for related peptides in the prior art.

If polyclonal antibodies are desired, a selected mammal, such as a mouse, rabbit, goat or horse, may be immunised with a polypeptide of the first aspect of the invention. The polypeptide used to immunise the animal can be derived by recombinant DNA technology or can be synthesized chemically. If desired, the polypeptide can be conjugated to a carrier protein. Commonly used carriers to which the polypeptides may be chemically coupled include bovine serum albumin, thyroglobulin and keyhole limpet hemocyanin. The coupled polypeptide is then used to immunise the animal. Serum from the immunised animal is collected and treated according to known procedures, for example by immunoaffinity chromatography.

Monoclonal antibodies to the polypeptides of the first aspect of the invention can also be readily produced by one skilled in the art. The general methodology for making monoclonal antibodies using hybridoma technology is well known (see, for example, references 90, 91 and 92.

Panels of monoclonal antibodies produced against the polypeptides of the first aspect of the invention can be screened for various properties, i.e., for isotype, epitope, affinity, etc. Monoclonal antibodies are particularly useful in purification of the individual polypeptides against which they are directed. Alternatively, genes encoding the monoclonal antibodies of interest may be isolated from hybridomas, for instance by PCR techniques known in the art, and cloned and expressed in appropriate vectors.

Chimeric antibodies, in which non-human variable regions are joined or fused to human constant regions (see, for example, reference 93), may also be of use.

The antibody may be modified to make it less immunogenic in an individual, for example by humanisation (see references 94, 95, 96, 97, 98 and 99). The term “humanised antibody”, as used herein, refers to antibody molecules in which the CDR amino acids and selected other amino acids in the variable domains of the heavy and/or light chains of a non-human donor antibody have been substituted in place of the equivalent amino acids in a human antibody. The humanised antibody thus closely resembles a human antibody but has the binding ability of the donor antibody.

In a further alternative, the antibody may be a “bispecific” antibody, that is an antibody having two different antigen binding domains, each domain being directed against a different epitope.

Phage display technology may be utilised to select genes which encode antibodies with binding activities towards the polypeptides of the invention either from repertoires of PCR amplified V-genes of lymphocytes from humans screened for possessing the relevant antibodies, or from naive libraries (see references 100 and 101). The affinity of these antibodies can also be improved by chain shuffling (see reference 102).

Accordingly, the present invention also provides antibodies obtained by the above techniques.

Antibodies generated by the above techniques, whether polyclonal or monoclonal, have additional utility in that they may be employed as reagents in immunoassays, radioimmunoassays (RIA) or enzyme-linked immunosorbent assays (ELISA). In these applications, the antibodies can be labelled with an analytically-detectable reagent such as a radioisotope, a fluorescent molecule or an enzyme.

### Expression of Membrane Proteins

High-level expression of integral membrane proteins at full-length is a useful tool for their structural and functional characterization {103}. However, the various eukaryotic host systems that have been developed for this purpose (based on, for example, yeast or mammalian cell lines) often involve cumbersome procedures and result in low protein yields. In contrast, over-expression in prokaryotes (particularly in Escherichia coli) is straightforward and has the potential to produce large quantities of recombinant protein. This is particularly useful when the recombinant protein is for use as a medicament, since large quantities of protein may be obtained at relatively low cost.

Unfortunately, high-level expression in E. coli of integral membrane proteins (i.e. proteins comprising hydrophobic transmembrane domains) is rarely possible because of the toxic effects of hydrophobic domains on the host cells. Accordingly, bacterial expression of membrane proteins has frequently been restricted to their soluble domains. This is not always desirable, however, as the missing transmembrane domains may contain important structural information directing the folding, oligomerization or subcellular sorting of the full-length membrane proteins.

Numerous efforts have been made to over-express eukaryotic membrane proteins in E. coli. These efforts have generally involved the use of specific fusion partners for the transmembrane domain of interest. For example, references 104, 105 and 106 describe the expression of various transmembrane domains within the context of tripartite fusion proteins consisting of the bacterial ToxR transcription activator domain, the particular transmembrane domain of choice and malE.

Accordingly, there remains a need in the art for a simple and generally applicable means of expressing membrane proteins, and in particular in prokaryotes, e.g. E. coli.

The present invention is also based on the surprising discovery that the fusion of an 18-mer peptide sequence (SEQ ID NO: 8), or a functional equivalent thereof, to a peptide comprising a transmembrane domain allows expression of said peptide.

Accordingly, in another aspect, the present invention provides a method for expressing a protein comprising one or more transmembrane domain(s) in an expression system, said method comprising the steps of:
a) fusing a sequence encoding an 18-mer peptide of SEQ ID NO: 8 or a functional equivalent thereof to a gene encoding said protein; and
b) expressing the resultant gene-fusion product in an expression system.

Suitable expression systems for this aspect of the invention are described under “Processes” above. Preferably the expression system is a prokaryotic expression system. Suitable prokaryotic expression systems for the present invention are again described above. Examples of particularly preferred prokaryotic expression systems include those that use streptococci, staphylococci, E. coli, Streptomyces and Bacillus subtilis as host cells. Preferably, the prokaryotic expression system is an E. coli expression system.

The present invention also provides a nucleic acid encoding SEQ ID NO: 8 or a functional equivalent thereof and vectors (e.g. plasmids) comprising said nucleic acid (e.g. expression vectors and cloning vectors).

In some embodiments, said nucleic acid is located in the vector next to one or more restriction sites. Preferably, said restriction sites are suitable for fusing a gene encoding a protein comprising one or more transmembrane domain(s) to said nucleic acid. In some embodiments, said nucleic acid is located in the vector next to one or more sequences that allow integration of nucleic acids into the vector. For example, said sequences may support integration of nucleic acid by homologous integration. Preferably, the sequences are suitable for fusing a gene encoding a protein comprising one or more transmembrane domain(s) to said nucleic acid.

In other embodiments, said nucleic acid is located in the vector such that it is fused to a gene encoding a protein comprising one or more transmembrane domain(s).

In particularly preferred embodiments, the vector comprises other nucleic acids encoding amino acid sequences that may be fused to a gene encoding the expression protein. Such sequences include leader sequences to direct protein trafficking, short peptide sequences which facilitate cloning or purification (e.g. histidine tags i.e. Hisₙ where n=3, 4, 5, 6, 7, 8, 9, 10 or more) or fluorescent tags. Particular examples include sequences for other proteins (e.g. MBP, thioredoxin, NusA, GST or GFP) or a sequences for an identifiable peptide (e.g. HA or c-Myc).

Other suitable amino acid sequences will be apparent to those skilled in the art.

The present invention also provides prokaryotic host cells transformed with such vectors. Preferably the host cells are E. coli.

In the methods and vectors described above, the sequence(s) encoding the transmembrane domain(s) in the gene encoding a protein comprising one or more transmembrane domain(s) may be at any position(s) within the gene. For example, the sequence(s) encoding the transmembrane domain(s) may be located at the 5′ or 3′, end(s) of the gene. Similarly, the sequence(s) may be positioned within the gene at (a) position(s) other than the 5′ or 3′ end(s). Where there is more than one sequence encoding a transmembrane domain, these sequences may be positioned independently of each other. Alternatively, they may be positioned in a particular arrangement. For example, the resultant protein may be a protein that can span a lipid membrane multiple times (e.g. a seven-transmembrane receptor, 7-TMR), wherein the transmembrane domains are arranged at specific intervals.

In preferred embodiments, at least one sequence encoding a transmembrane domain is located at the 3′ end of the gene encoding the protein comprising one or more transmembrane domain(s). In other preferred embodiments, at least one sequence encoding a transmembrane domain is located at the 5′ end of the gene encoding the protein comprising one or more transmembrane domain(s).

Preferably, the protein comprises one transmembrane domain.

In the methods and vectors described above, the sequence encoding the 18-mer peptide or a functional equivalent thereof may also be at any position relative to the gene encoding the protein comprising a transmembrane domain. For example, the sequence encoding the 18-mer peptide or a functional equivalent thereof may be fused to the 5′ or 3′ end of the gene. Similarly, the sequence may be fused within the gene at a position other than the 5′ or 3′ end.

In preferred embodiments, the sequence encoding the 18-mer peptide or a functional equivalent thereof is fused to the 3′ end of the gene encoding the protein comprising one or more transmembrane domain(s). This embodiment is particularly preferred when at least one sequence encoding a transmembrane domain is located at the 3′ end of the gene encoding the protein comprising one or more transmembrane domain(s). In other preferred embodiments, the sequence encoding the 18-mer peptide or a functional equivalent thereof is fused to the 5′ end of the gene encoding the protein comprising one or more transmembrane domain(s). This embodiment is particularly preferred when at least one sequence encoding a transmembrane domain is located at the 5′ end of the gene encoding the protein comprising one or more transmembrane domain(s).

When the gene encoding the protein comprising one or more transmembrane domain(s) comprises at least one sequence encoding a transmembrane domain at a position other than the 5′ or 3′ end, the sequence encoding the 18-mer peptide or a functional equivalent thereof is preferably fused to the 5′ or 3′ end of that sequence.

In some embodiments, the gene encoding the protein comprising a transmembrane domain is a heterologous gene, preferably an eukaryotic gene.

### General

The term “comprising” encompasses “including” as well as “consisting of” e.g. a composition “comprising” X may consist exclusively of X or may include something additional e.g. X+Y.

The term “about” in relation to a numerical value x means, for example, x±10%.

The word “substantially” does not exclude “completely” e.g. a composition which is “substantially free” from Y may be completely free from Y. Where necessary, the word “substantially” may be omitted from the definition of the invention.

The term “functional equivalent”, as used herein, refers to a sequence that has an analogous function to the sequence of which it is a functional equivalent. By “analogous function” is meant that the sequences share a common function and, in some embodiments, a common evolutionary origin. For example, where the sequence is derived from gp41 from HIV-1 strain HXBR2, functional equivalents would include sequences derived from genes encoding evolutionarily-related proteins in other HIV-1 clades/strains, and viruses related to HIV-1 (and clades/strains thereof) including, but not limited to, HIV-2 and SIV. In some embodiments, a functionally equivalent sequence may exhibit sequence identity with the sequence of which it is a functional equivalent. Preferably, the sequence identity between the functional equivalent and the sequence of which it is a functional equivalent is at least 50% across the length of the functional equivalent. More preferably, the identity is at least 60% across the length of the functional equivalent. Even more preferably, identity is greater than 70%, 80%, 85%, 90%, 95%, 96%, 97%, 98% or 99% across the length of the functional equivalent. Functional equivalents include mutants of the sequences of which they are functional equivalents, i.e. containing amino acid substitutions, insertions or deletions from said sequence, provided that function is retained. Functional equivalents with improved function compared to the sequences of which they are functional equivalents may be designed through the systematic or directed mutation of specific residues in said sequences. Functional equivalents include sequences containing conservative amino acid substitutions that do not affect the function or activity of the sequence in an adverse manner.

References to a percentage sequence identity between two amino acid sequences means that, when aligned, a percentage of the amino acids are the same in the two sequences. This alignment and the percent homology or sequence identity can be determined using software programs known in the art, for example those described in section 7.7.18 of reference 107. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in reference 108.

The use of “NH₂” and “COOH” in peptide sequences implies only the direction of the peptide chain from N-terminus to C-terminus, and does not imply that the N-terminus residue must have a free —NH₂ group or that the C-terminus must have a free —COOH group (although nor is such a situation excluded). On the contrary, the N- and C-termini may be covalently modified.

### BRIEF DESCRIPTION OF DRAWINGS

FIG. 1 shows a schematic drawing of gp41 and gp41ctm. The N-terminal heptad repeat region is designated as HR1 and the C-terminal heptad repeat region as HR2. Gp41Nflag is also indicated. The sequences of inhibitory peptides C34 and T-20 and of 2F5 and 4E10 antibody epitopes are marked. For the 2F5 antibody epitope, the core epitope (solid line) and the extended eptiope (dashed line) are shown. “FP” refers to fusion peptide; “TM” refers to transmembrane domain and “N18” refers to 18-mer (SEQ ID NO: 8).

FIG. 2 shows the oligomerization and stability of gp41ctm in solution and incorporated into liposomes. (A) Soluble gp41ctm (PBS, 1% β-OG) was incubated with increasing concentrations of EGS (lane 1, no EGS; lane 2, 0.1 mM; lane 3, 0.25 mM; lane 3, 0.5 mM; lane 4, 1 mM; lane 5, 5 mM). (B) Sucrose gradient purified gp41ctm liposomes were incubated with EGS (lane 1, no EGS; lane 2, 1 mM; lane 3, 5 mM). The samples were separated on a reducing SDS-PAGE followed by Coomassie brilliant blue staining. Gp41ctm monomers and putative dimers and trimers are indicated by numbers. (C) Protease digestion of gp41ctm and (D) of gp41ctm incorporated into liposomes. Trypsin and chymotrypsin were used at the concentrations (w/w) indicated. Samples were analyzed by SDS-PAGE followed by Coomassie brilliant blue staining. The fuzzy broad band (arrow) migrating in (D) at approximately 6 kDa corresponds to lipids.

FIG. 3 shows the circular dichroism spectra of gp41ctm. (A) The two minima at 208 and 222 nm are characteristic for a-helical content. Spectra were measured in PBS, PBS 1% β-OG and H₂O. (B) Thermal denaturation curves of gp41ctm recorded at 222 nm in the same buffers as used in A. The measured ellipticity was converted to molar ellipticity.

FIG. 4 shows gp41ctm binding to gp41Nflag and mAbs 2F5 and 4E10. (A) Gp41ctm pull down of gp41Nflag. Gp41Nflag, gp41ctm and potential complexes formed by gp41ctm and gp41Nflag were passed over an anti-flag column, washed and bound protein eluted by low pH treatment. This shows elution of gp41Nflag (lane 1), no binding of gp41ctm (lane 2) and elution of a complex formed by gp41ctm and gp41Nflag (lane 3). Samples were separated on SDS-PAGE and bands were visualized with Coomassie brilliant blue staining. (B) Gp41ctm containing proteoliposomes were purified over a sucrose gradient (gp41ctm) and then incubated with either mAb 2F5 or 4E10 and subjected to a further round of sucrose gradient purification to remove unbound mAbs. The upper fraction of the gradient contains both gp41ctm and either mAb 2F5 or 4E10 as indicated. “lc” refers to antibody light chain; “hc” refers to antibody heavy chain. Samples were analyzed using SDS-PAGE followed by Coomassie brilliant blue staining. Only the sample from the top of the gradient is shown.

FIG. 5 shows gp41ctm inhibiting HIV-1 entry. Gp41ctm alone, gp41ctm containing liposomes, the HIV fusion inhibitor T-20 and empty liposomes as a negative control were added in the concentration indicated together with modified HIV virus to TZM-b1 cells. Two days later, infection cells were lysed and expression of luciferase activity measured. The Y-axis shows the luciferase activity and the X-axis indicates in a logarithmic scale the concentration of the compounds added. Each Experiment was run in triplicate and repeated with 3 different gp41ctm liposome preparations.

FIG. 6 shows the immunogenicity of gp41ctm. (A) Serial dilution of sera from mice immunized with gp41ctm alone or gp41ctm-liposomes was incubated with gp41ctm coated onto ELISA plates. Bound antibodies were detected with anti mouse IgG1, IgG2a and IgA antibodies followed by incubation with horseradish peroxides conjugated secondary antibodies. The emitted signal was measured at 450 nm after the addition of TMB. The letters at the X-axis indicate individual mice. The Y-axis shows the reciprocal titer of the antibodies. “gp41ctm-lip” refers to gp41ctm incorporated into liposomes. (B) shows the neutralizing activities of mice sera. Serial dilution of sera from mice immunized with empty liposomes or gp41ctm-liposomes or gp41ctm alone were analysed in a syncytium inhibition assay. Sera were preincubated with tissue line adapted HIV-1 RF virus before addition of AA-2 cells. “Lip” refers to empty liposomes.

FIG. 7 shows the elution profile of chrtm5 on a gel filtration column. Chrtm5 was overexpressed in E. coli and purified on a Superdex 200 gel filtration column in purification buffer (25 mM HEPES pH 7.4, 0.1 M KCl) containing 1% beta octyl-glucopyranoside.

FIG. 8 shows chemical cross-linking of chrtm5. Chrtm5 was cross-linked with sulfo-ethylene glycol bis(succinimidylsuccinate) (S-EGS). Lane 1—marker; Lane 2—pure chrtm5; and Lane 3—cross-linked CHRTM5.

FIG. 9 shows a pull down assay with mAbs 2F5 and 4E10. Lane 1—marker; Lane 2—control without antibody; Lane 3—mAb 2F5; and Lane 4—mAb 4E10.

FIG. 10 shows the elution profile of a chrtm5/2F5 Fab complex. Chrtm5/2F5 Fab complexes were formed and purified on a Superdex 200 gel filtration column.

FIG. 11 shows an SDS-PAGE analysis of a chrtm5/2F5 Fab complex. Fractions corresponding to the major peak shown in FIG. 10 were separated by SDS-PAGE and stained with Coomassie brilliant blue.

FIG. 12 shows a Western blot analysis of rabbit serum generated by chrtm5-proteoliposome immunization. The oligomeric forms of CHRTM5 are indicated by numbers. The same pattern was obtained with serum dilutions of 1:50, 1:100 and 1:1000.

### MODES FOR CARRYING OUT THE INVENTION

### HIV-1

The following Examples demonstrate how the present invention may be applied to HIV-1 gp41, more specifically, HIV-1 gp41 from the strain HXB2R. However, from the teachings given herein, it would require no more than routine experimentation for the skilled person in the art to apply the following examples to any other protein that comprises a domain that is a functional equivalent of the gp41 HR2 domain.

### EXAMPLE 1

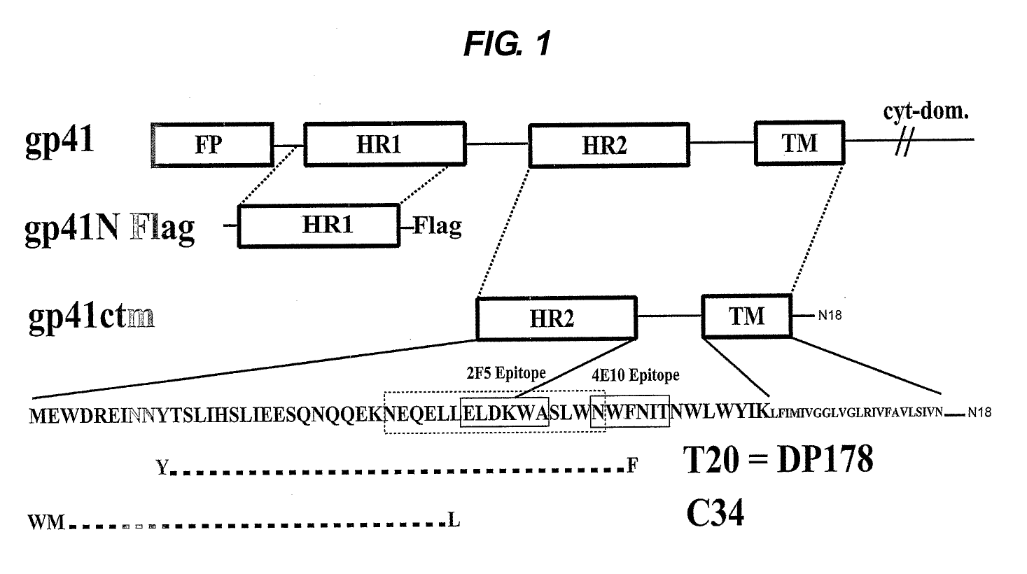

The present inventors have created a gp41 construct (gp41ctm) comprising the Env transmembrane domain and the extracellular C-terminal region, including peptide regions that have been shown to exert fusion inhibition (e.g. T-20 and C34) (FIG. 1). The Env transmembrane domain constitutes a trimerization domain. Trimeric gp41ctm is protease resistant and recognized by mAbs 2F5 and 4E10. It also exerts potent anti-viral activity either in solution or when incorporated into liposomes. Initial immunization studies in mice indicate low immunogenicity of gp41ctm in solution while liposome-incorporated gp41ctm generates IgG and IgA responses that show neutralization capacity.

### Materials and Methods

Expression constructs. HIV-1 (strain HXB2R) gp41 cDNA (nucleic acid position 1885 to 2118; gp41 residues 118-195) was amplified by standard PCR and cloned into the expression vector pRSET (Invitrogen). DNA sequencing revealed the C-terminal addition of 18 amino acids encoded by the vector sequence. The resultant construct was designated gp41ctm (SEQ ID NO: 14). HIV-1 gp41 cDNA (nucleotides 1605-1743; gp41 residues 24 to 70) (gp41-Nflag; N-terminal helix) were amplified by PCR with the addition of a C-terminal Flag tag and cloned into a pRSET expression vector. The sequence was confirmed by DNA sequencing.

Protein purification. Gp41ctm and gp41N were expressed in E. coli host strain BL21 (DE3) pUBS. Expression of the proteins was induced with 1 mM isopropyl-13-D-thiogalactopyranoside (IPTG) for three hours at 37° C. Gp41ctm expressing cells were lysed by sonication in buffer A (50 mM Tris pH 8, 100 mM NaCl, 1% CHAPS). The cleared supernatant was loaded onto a Q sepharose column in buffer A and gp41ctm was eluted by applying a 0.1-1M NaCl gradient. Gp41ctm containing fractions were pooled and dialyzed against H₂O, subsequently adjusted to buffer A and subjected to one more Q-sepharose purification step. Final purification included separation on a superdex 200 column (Amersham Biosciences) in buffer B (20 mM Hepes pH 8, 100 NaCl, 1% β-octylglucopyranoside). Lysates from cells expressing gp41Nflag were loaded onto a S-Sepharose column in 50 mM Tris pH8, 0.1M NaCl and eluted by applying a 0.1 to 1 M NaCl gradient. Fractions containing gp41N-flag were identified by SDS-PAGE and used for pull-down experiments.

CD-Analysis. The CD spectrum of gp41ctm (0.16 mg/ml) in 10 mM phosphate pH 7.2, 50 mM NaCl was recorded at 20° C. using a 1-mm cell on an a Jasco J-810 spectropolarimeter equipped with a thermoelectric controller. The thermodynamic stability was measured at 222 nm by monitoring the CD signal in the temperature range of 4-98° C. The measured signal was then converted into molar ellipticity.

Chemical Cross-linking. Gp41ctm (PBS 1% β OG) and gp41ctm incorporated into liposomes (PBS) were incubated with ethyleneglycol bis(-succinimidylsuccinate) EGS (Pierce) as indicated for 20 min at room temperature. The reaction was quenched by the addition of 50 mM Tris, pH 8.0, for 5 min. Samples were separated by SDS-polyacrylamide gel electrophoresis under reducing conditions, and the bands were visualized by Coomassie Brilliant Blue staining.

Liposome preparation and flotation assay. Gp41ctm proteoliposomes (25% L-alpha-phosphatidylcholine, 50% L-alpha-phosphatidyl-L-serine, 25% cholesterol) preparation was modified as previously described {109}. Briefly, lipid films were resuspended with gp41ctm in buffer A and the detergent was removed by dialysis with a liposomat (Dianorm). The proteoliposomes were adjusted to 50% Sucrose (in PBS), overlaid with 40%, 30%, 10% and 0% Sucrose (in PBS) and centrifuged in a SW 41 rotor at 40 000 rpm for 12 hours. Fractions from the sucrose gradients were collected and samples were separated on a 12% SDS-PAGE. Fractions containing gp41ctm bands were identified by Coomassie Blue staining. After incubation (15 min at RT) of gp41ctm proteoliposomes with either mAb 2F5 or 4E10 similar sucrose gradient centrifugation analyses were applied to separate free mAb and gp41ctm/mAb proteoliposomes. Samples of fractions containing gp41ctm/mAb proteoliposomes were separated on 12% SDS-PAGE and stained with Coomassie Brilliant Blue.

For the immunization studies, the liposomes were produced by the crossflow injection technique using a multiple injection mode ({10} and {111}). Briefly, the lipid mixture is dissolved in 95% ethanol at 50° C. and the temperature of the protein/detergent solution is equilibrated at 50° C. at a β-OG concentration of 1%. For vesicle formation, the protein/detergent solution is pumped from container A to container B and, while pumping the micellar protein solution through the crossflow injection module, the ethanol/lipid solution is injected followed by immediate dilution with PBS in vessel B. After dilution, the β-OG concentration is reduced to 0.1% resulting in a stable liposome solution. The successful incorporation of gp41ctm was confirmed by western blot and FACS analysis using 2F5 mAbs.

Pull down assay. N-Flag and gp41ctm were incubated for 30 min at RT in buffer B, diluted 1:2 in TBS/1% Triton and loaded onto a Flag-Agarose column, washed extensively with TBS/1% Triton and eluted with 0.1M Glycine/HCl pH 3.5. Proteins were analyzed by SDS-PAGE and visualized by Coomassie Brilliant Blue staining.

HIV Neutralization assay. TZM-b1 cells, a HeLa cell line derivative expressing CD4, CXCR4 and CCR5 and firefly luciferase upon infection with HIV, were seeded at a density of 3×10³ cells per 96-well plate (43). The next day, cells were pre-treated with T-20, gp41ctm and liposomes with and without incorporated gp41ctm at different concentrations for 30 minutes at room temperature. 1000 infectious units per well (as determined on TZM-b1 cells) of HIV_{NL-4.3} were used to challenge the cells and 2 days later the cells were lysed and the activity of firefly luciferase activity determined (Steady-Glo luciferase system, Promega, Germany). Due to the induction of firefly luciferase upon infection, the reduction of relative light units (RLU) detectable correlates with the inhibition of infection due to T-20, gp41ctm and liposomes with and without incorporated gp41ctm, respectively. The viability of the cells was not affected by the addition of gp41ctm or gp41ctm-liposomes.

Immunization. BALB/c inbred female mice 6-8 weeks of age were immunized intraperitoneally (i.p.). Six mice per group were primed with gp41ctm or with gp41ctm proteoliposomes. The second immunization was performed three weeks later with the same preparations. Control groups were immunized with an empty liposome preparation utilizing the same immunization procedures. Two weeks following the boost mice were bled from Sinus orbitalis and sera were stored at −20° C. before further analysis.

Enzyme linked immunosorbent assay (ELISA). An ELISA protocol was performed as described in reference 20 utilizing gp41ctm protein (2.5 μg/ml carbonate buffer, pH 9.6) as coating antigen. Serial dilutions of sera in PBS/Tween containing 1% skim milk were added to the coated plates and the mixtures incubated for 1.5 h at room temperature. Bound antibodies were detected with goat anti-mouse IgG1- and IgG2a (gamma-chain-specific) conjugated with horseradish peroxidase (Zymed). Following additional washing steps plates were stained with 3,3′,5.5′-tetramethylbenzidine as substrate. The reaction was stopped with 1.25 M H₂SO₄ and the plates were measured (wavelength 450 nm). The cut-off value is defined as the mean value of absorption of serum samples of mice immunized with empty liposomal preparations plus two standard deviations.

Syncytium Inhibition Assay. Inhibition of HIV-1 replication by serum samples was assessed in a standard syncytium inhibition assay. Experiments were performed with sera from mice immunized with gp41ctm or gp41ctm-containing liposomes. Sera from mice, which had received liposomes only were run in parallel as negative controls. AA-2 cells were used as an indicator cell line with syncytium formation as read-out. Briefly, ten serial two-fold dilutions of serum samples in cell culture medium (RPMI-1640, 10% FCS, 4 mM L-Gln, 5 μg/ml polybrene) were pre-incubated with 10²-10³ TCID₅₀ of tissue culture line adapted HIV-1 RF for 1 h at 37° C. before addition of AA-2 cells. Cells were incubated for 5 days before assessment of syncytium formation. Experiments were performed with 4 replicates per dilution step. The presence of at least one syncytium per well was considered as indication for HIV-1 infection. The 50% inhibiting titer was calculated according to the method of Reed and Muench ({112}). Unspecific inhibition by control sera was used as cut-off. All assays included a virus titration of the inoculum to confirm the infectious titer.

### Results

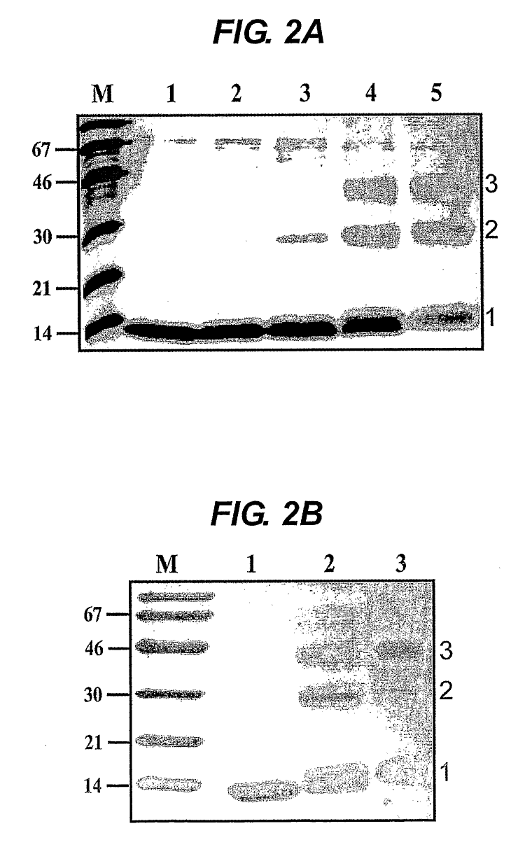

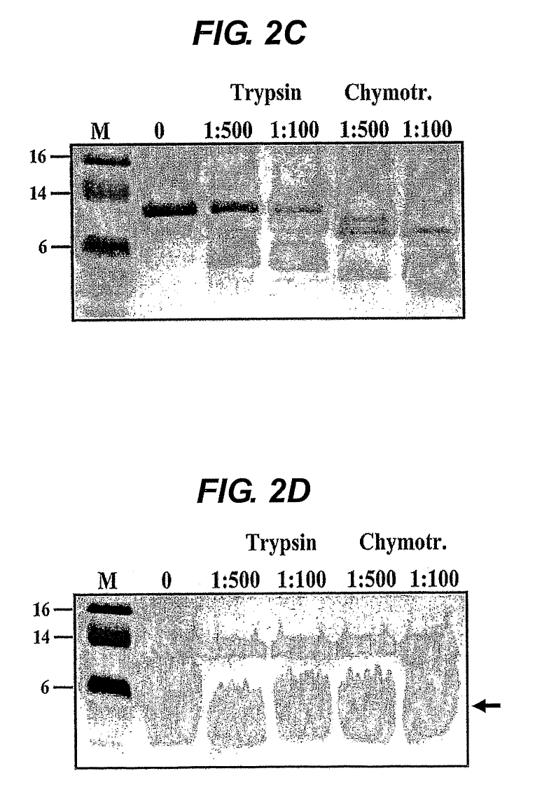

Oligomerization and protease sensitivity of gp41ctm. Due to bacterial toxicity, initial attempts to express gp41 residues 118 to 195, comprising a sequence derived from the HR2 domain, the transmembrane region and the sequence connecting these two domains failed. However, a clone was selected which has an extended 3′ open reading frame that includes 18 residues (SEQ ID NO: 8) and allows bacterial expression. The resultant construct was designated gp41ctm. Gp41ctm can be purified to homogeneity and is monodisperse in solutions containing 1% detergent (β-OG or CHAPS). It forms soluble aggregates in just PBS or H₂O, but elutes from a gel filtration column at approximately 14 ml in PBS containing 1% CHAPS or 1% β-OG indicating a distinct oligomeric state. The elution volume may also indicate an extended conformation of gp41ctm since a 50-kDa marker protein (antibody Fc fragment) elutes at approximately 15 ml and a 220 kDa protein (catalase) elutes at approximately 12 ml under the same conditions. Chemical cross linking of gp41ctm in detergent (FIG. 2A) and gp41ctm incorporated into liposomes (FIG. 2B) with increasing concentrations of EGS cross linking reagent gives raise to a second band migrating at approximately 30 kDa and a third band migrating at approximately 45 kDa in both experiments (FIG. 2). This is consistent with the trimeric nature of the envelope glycoprotein ({113} and {114}) and indicates that the transmembrane region is the minimal trimerization domain of HIV-1 Env gp160.

In order to test the presence of structural features, gp41ctm was subjected to protease digestion either alone or incorporated into liposomes. This shows cleavage of gp41ctm at high protease concentrations resulting in two smaller trypsin bands migrating at approximately 9 and approximately 4 kDa as well as two slightly smaller chymotrypsin fragments migrating at approximately 8 and approximately 3 kDa (FIG. 2C). In contrast, gp41ctm incorporated into liposomes was not cleaved under the same conditions, indicating increased resistance (FIG. 2D).

The protease resistance of liposome-incorporated gp41 ctm makes it particularly well suited for topical administration.

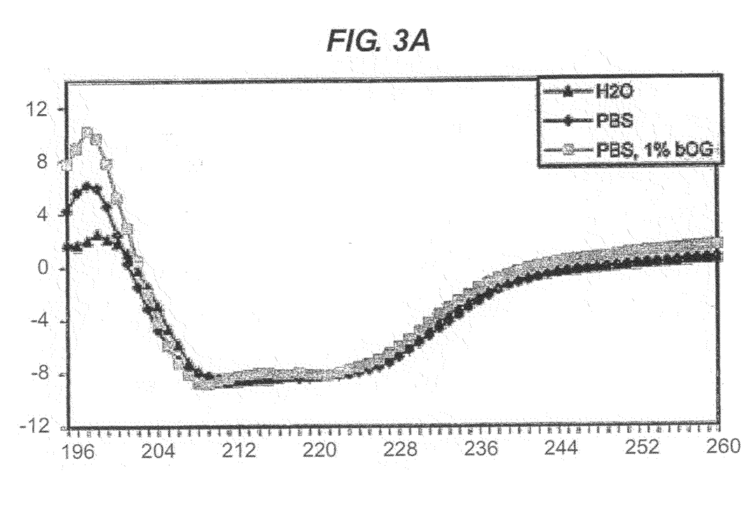

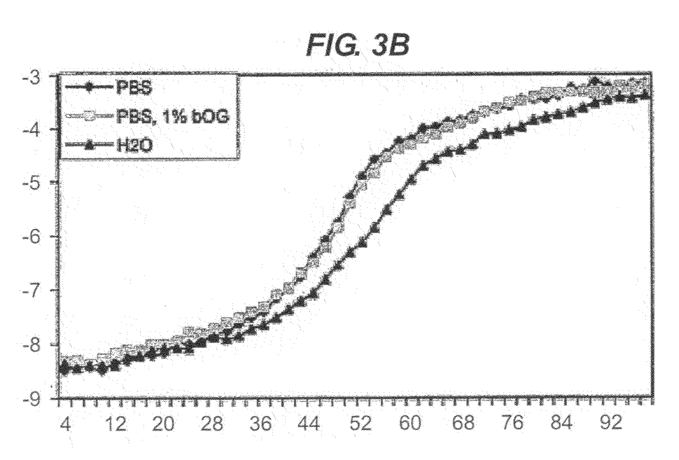

CD analysis of gp41ctm. Secondary structure analysis of gp41ctm by circular dichroism reveals a-helical spectra with a calculated a-helical content of approximately 30%. This is independent of the buffer conditions as the same spectra were obtained in buffers containing H₂O, PBS, or PBS containing 1% β-OG (FIG. 3A). The thermal stability of gp41ctm is indistinguishable in PBS and in PBS containing 1% 13-OG and shows somewhat greater stability in H₂O. The unfolding transition of gp41ctm occurs at approximately 48° C. in PBS buffers and at approximately 52° C. in H₂O (FIG. 3B).

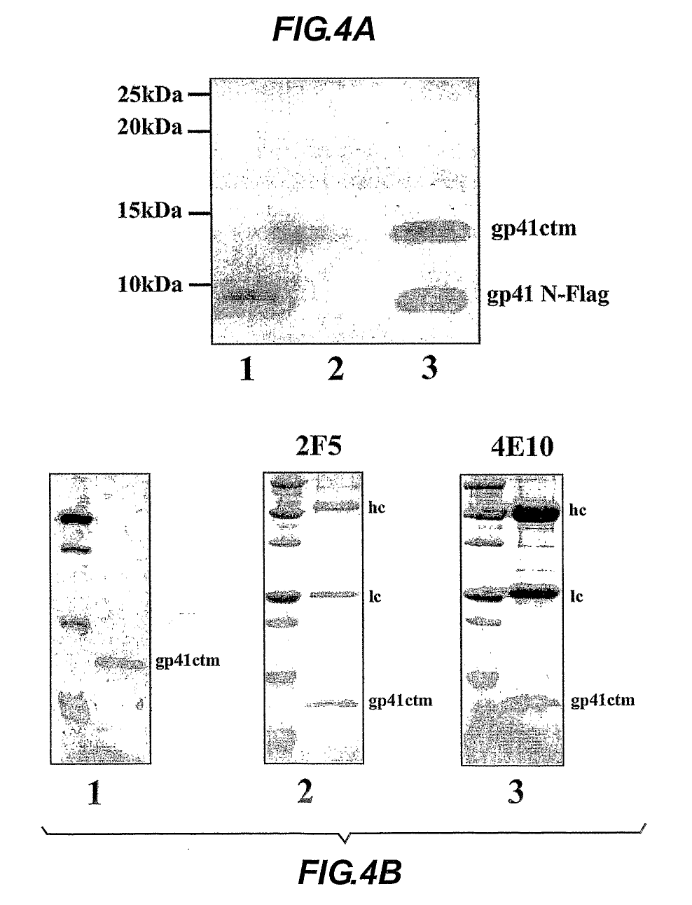

Gp41ctm binding to the gp41 N-terminal helix. Gp41ctm contains the C-terminal helical region (HR2), which interacts with the N-terminal triple stranded coiled coil region (HR1) in the gp41 core structure (FIG. 1). In order to show that the C-terminal helical region of gp41ctm is available for binding to gp41 HR1, a pull down assay was performed with flag-tagged gp41Nflag. As expected, gp41Nflag bound to the column and could be eluted (lane 1), while gp41ctm did not bind (lane 2). However, when gp41Nflag and gp41ctm were preincubated prior to column binding, the complex composed of both fragments was eluted. This indicates that the C-terminal helix is available to interact with the N-terminal helical construct gp41Nflag (FIG. 4A; lane 3).

Gp41ctm interaction with mAbs 2F5 and 4E10. Gp41ctm contains two membrane proximal epitopes (FIG. 1) that are recognized by two broadly neutralizing human antibodies, namely mAb 2F5 and 4E10.

It was therefore analyzed whether these mAbs recognize trimeric gp41ctm in a membrane environment. Gp41ctm was incorporated into liposomes, incubated with mAbs and free mAbs were separated by sucrose density gradient centrifugation. This revealed that both mAbs 2F5 and 4E10 bound to gp41ctm and were found in the upper fraction of the gradient (FIG. 4B) similar to the floatation of gp41ctm liposomes alone (FIG. 4B), whereas antibodies incubated with empty liposomes remain in the loading zone of the gradient. This indicates that gp41ctm is incorporated into the liposomes correctly in that the extracellular region is accessible for mAbs 2F5 and 4E10 interactions.

It was also analyzed whether Fab fragments of these mAbs recognize trimeric gp41ctm in solution. Gp41ctm was purified and eluted from a gel filtration column (superdex 200) in a buffer containing 20 mM Hepes pH 8.0, 100 mM NaCl and 1% beta-OG. Fab fragments were generated from mAbs 2F5 and 4E10 by papain cleavage following standard protocols. Purified gp41ctm was then incubated with an excess of Fab 2F5 or Fab 4E10 and passed over a gel filtration column (superdex 200) in the same buffer. Fractions containing gp41ctm and Fabs were identified by SDS PAGE analysis. Gp41ctm-2F5 complexes eluted at approximately 12 ml, while gp41ctm incubated with Fab 4E10 eluted at the same position as gp41ctm alone (approximately 14 ml). This shows that gp41ctm is capable of forming a complex with Fab 2F5 in solution, but is not capable of forming a complex with Fab 4E10 in solution.

In contrast, the ability of gp41ctm to react with mAb 4E10 when inserted into liposomes suggests that a bilayer environment is important for mAb 4E10 epitope presentation.

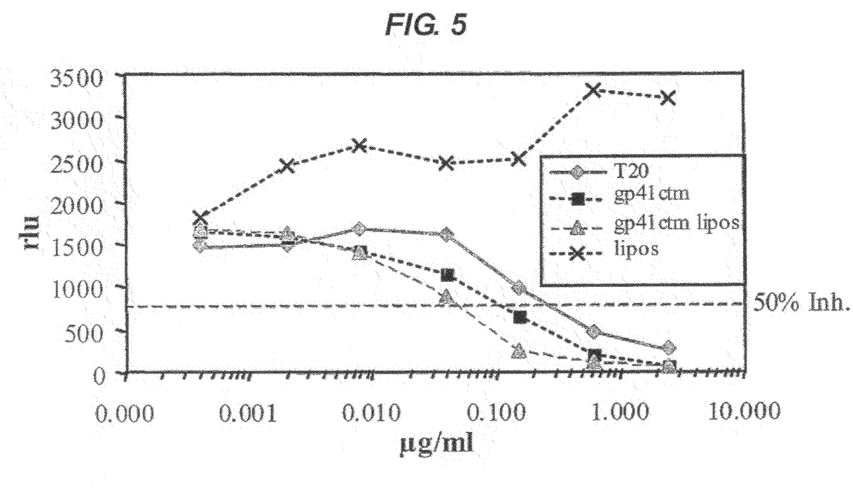

Antiviral activity of gp41ctm. Gp41ctm contains peptide sequences, which had been previously shown to exert potent anti-viral activity such as T-20 and C34 (FIG. 1). These C-terminal peptides interact with the N-terminal coiled coil of gp41 thus blocking the refolding of gp41 into the six helical bundle structure ({115}, {116}. {117} and {39}). Using a sensitive single round infection assay that measures infection based on the stimulation of luciferase activity {43}, it was found that soluble gp41ctm has a strong inhibitory effect with an IC50 of 0.4 μg/ml (34 nM), which is around two times more efficient then the IC50 of 0.2 μg/ml (45 nM) calculated for T-20 using this assay. Surprisingly, gp41ctm incorporated into liposomes had an even stronger inhibitory effect on HIV-1 than soluble gp41ctm alone with an IC50 of 0.15 μg/ml (12 nM). Empty liposomes tested as a control showed no inhibitory activity (FIG. 5). Together, these data indicate that soluble gp41ctm or membrane-anchored gp41ctm has a potent anti-viral activity that is greater than therapeutic fusion inhibitors such as T-20.

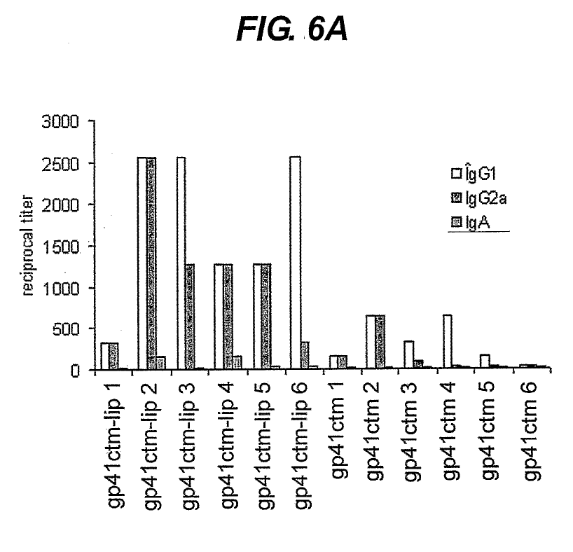

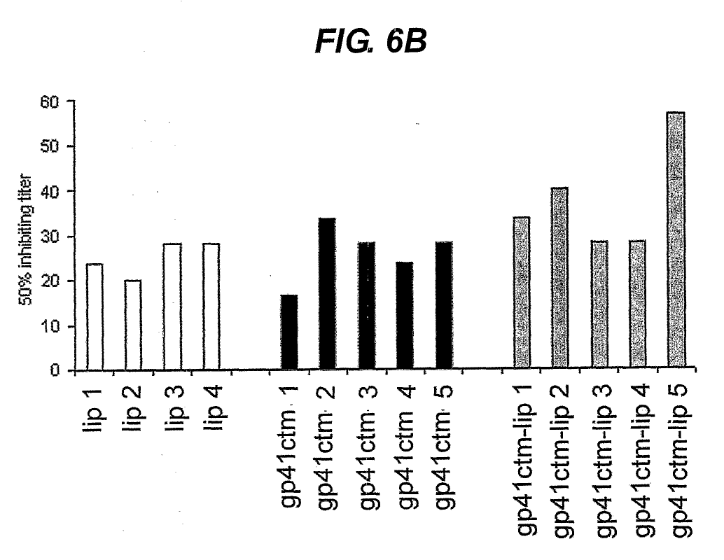

Immunogenicity of gp41ctm. It was have shown that gp41ctm binds to mAbs 2F5 and 4E10 in its soluble form as well as when membrane anchored. Although these antibodies can be mapped to linear gp41 epitopes (FIG. 1) the nature of the antigen which is capable of inducing such an immune response remains elusive ({23}). The immunogenicity of trimeric gp41 in solution and when incorporated into liposomes was therefore tested. Mice were immunized intraperitoneally (i.p.) with gp41ctm liposomes or with gp41ctm alone or, as a control, with empty liposomes. After two rounds of immunization, the mice were bled and the reactivity of the sera against gp41ctm was tested using a standard ELISA protocol (FIG. 6A). All mice immunized with gp41ctm liposomes showed a strong IgG1 and IgG2a specific immune response against gp41ctm up to a reciprocal titer of 2560. In contrast, only 3 mice out of 5 immunized with soluble gp41ctm showed a weak response with reciprocal titers between 250 and 640 (FIG. 6A). Although low reciprocal titers for IgA (approximately 150) could be detected in 2 out of 5 mice immunized with gp41ctm liposomes, no IgA response could be determined in the gp41ctm group (FIG. 6A). This indicates that gp41ctm has poor antigenicity by itself. However, when incorporated into liposomes, gp41ctm has high antigenicity even after only two rounds of immunization.

The sera were then tested for their neutralizing activity using a syncytium inhibition assay. Sera from the control group (liposomes only) showed unspecific inhibition of syncytium inhibition up to a titer of 1:28.2. This is a common effect of mouse sera and was within the usual range. In contrast, two gp41ctm liposome derived sera neutralized the HIV-1 RF strain at a higher titer than observed in the control sera (1:33.6 and 1:40). A third serum derived from gp41ctm liposome immunization showed a very much higher titer of 1:57 (FIG. 6B). This suggests that gp41ctm incorporated into liposomes is a promising vaccine candidate as it is both immunogenic and elicits the production of neutralizing antibodies.

In contrast, only one serum from the group immunized with soluble gp41ctm showed a neutralizing titer, and this was only slightly above the control group (FIG. 6B). This suggests that soluble gp41ctm is particularly well suited for use as a fusion inhibitor as few or no neutralizing antibodies, which might inhibit its activity, would be produced in vivo.

### EXAMPLE 2

The present inventors have also created an HIV-1 gp41 construct comprising a soluble oligomerization domain (a trimeric version of the 30 amino acid coiled coil in GCN4) and part of the extracellular C-terminal region, which includes the peptide region comprising the 4E10 epitope and the extended 2F5 epitope (SEQ ID NO: 18). The soluble oligomerization domain constitutes a trimerization domain. The trimeric construct is recognised by mAbs 2F5 and 4E10, confirming functional 2F5 and 4E10 epitope presentation.

### Materials and Methods

Expression of a gp41 sequence fused to a trimeric coiled coil region derived from the transcription factor GCN4. HIV-1 (strain HXB2R) gp41 cDNA (gp41 residues 143-171) was fused to a nucleic acid encoding SEQ ID NO: 7 by standard PCR methods and cloned into bacterial expression vector pPROEX HTb (Invitrogen), retaining an N-terminal His-tag. The fusion protein was expressed in E. coli cells BL21 codon⁺ (Invitrogen) and the cells lysed in a buffer containing 50 mM Tris pH 8 and 100 mM NaCl. The fusion protein was purified on a Ni²⁺ affinity column using standard procedures. The His-tag was cleaved off with TEV protease and the resultant peptide (SEQ ID NO: 18) further purified on a Superdex 200 gel filtration column in a buffer containing 20 mM Tris pH 8.0 and 1.00 mM NaCl.

### Results

Interaction with mAbs 2F5 and 4E10. Trimerization was confirmed by chemical cross-linking, gel filtration and dynamic light scattering. Complex formation of the peptide with Fabs generated from mAb 2F5 and mAb 4E10 (using standard protocols) showed that three Fabs from either 2F5 or 4E10 bound. This indicates functional 2F5 and 4E10 epitope presentation.

### EXAMPLE 3

The present inventors have also created a gp41 construct (gp41int) comprising an N-terminally elongated peptide of the invention (SEQ ID NO: 19). The N-terminal elongation comprises an N-terminal methionine residue, a His-Tag and a soluble oligomerization domain (a trimeric version of the 30 amino acid coiled coil in GCN4). The construct is recognised by mAbs 2F5 and 4E10, confirming functional 2F5 and 4E10 epitope presentation.

### Materials and Methods

Expression of N-terminally elongated peptide of the invention. The trimeric version of the 30 amino acid coiled coil in GCN4 (SEQ ID NO: 7), further comprising an N-terminal methionine residue and His-tag, was fused to gp41 residues 119-195, further comprising a C-terminal 18-mer (SEQ ID NO: 8) and cloned into pRSET. The resultant construct was designated gp41int (SEQ ID NO: 19). Gp41int was expressed in BL21 codon⁺ E. coli cells. Cells were lysed in buffer containing 50 mM Tris pH 8.0, 100 mM NaCl and 0.5% Triton and the gp41int purified on a Ni²⁺ sepharose column using standard procedures. Before elution of gp41int, Triton was exchanged with either 1% β-OG or 1% CHAPS in 50 mM Tris pH 8.0, 100 mM NaCl. Gp41int was then incorporated into liposomes as described for gp41ctm in “Liposome preparation and flotation assay” above.

### Results

Interaction with mAbs 2F5 and 4E10. Gp41int proteoliposomes reacted with mAbs 2F5 and 4E10. Gp41int proteoliposomes in complex with mAb 2F5 and 4E10 were purified by sucrose gradient centrifugation as described above.

A C-terminally truncated form of gp41int (lacking the C-terminal 18 mer of SEQ ID NO: 8) could not be expressed in E. coli.

### EXAMPLE 4

The present inventors have also created a further gp41 construct (chrtm5) comprising the Env transmembrane domain and the extracellular C-terminal region, including peptide regions that have been shown to exert fusion inhibition (e.g. T-20 and C34). Chrtm5 is a suitable gp41 antigen to potentially induce immune responses that produce neutralizing antibody activities similar to those described for the broadly neutralizing antibodies 2F5 and 4E10 directed against membrane proximal epitopes in gp41 present in chrtm5.

### Materials and Methods

Expression constructs. HIV-1 HXBR2 gp41 nucleotides corresponding to nucleic acid position 1885 to 2133 (gp41 residues 118 to 200) were amplified by standard PCR and subcloned into the pETM11plasmid (EMBL Protein Expression Facility). This gp41 construct contains an N-terminal HIS-tag, a TEV protease site, an Enterokinase protease site, the gp41 HR2 region, epitopes for antibodies 2F5 and 4E10, the gp41 transmembrane region and five residues from the cytoplasmic domain of gp41. An N-terminal Nco I and a C-terminal Acc65 I allow to use the sequence within the pETM series of vectors available from the EMBL protein expression facility.

Protein purification. For chrtm5 expression, the pETM11 plasmid was transformed into BL21(DE3)C41 E. coli cells. Expression is induced by IPTG (concentration of 0.2 mM) for 16 hours at 25° C. Cells are harvested at 15000×g for 15 min at 4° C. and resuspended in lysis buffer (20 mM Tris pH 8, 0.1 M NaCl, 1% CHAPS). Cell lysis was performed by ultrasonication for 5 min. Cell debris was pelleted at 48000×g for 45 min at 4° C. The supernatant was loaded onto a Ni-chelating sepharose column in lysis buffer. Chrtm5 was eluted with a 50-500 mM imidazole gradient in elution buffer (20 mM Tris pH 8, 0.1 M NaCl, 1% CHAPS). Fractions containing chrtm5 were pooled and dialyzed against purification buffer (25 mM HEPES pH 7.4, 0.1 M KCl, 1% (w/v) β-OG). Final purification was performed on a HiPrep 16/60 Sephacryl 200 or a Superdex 200 column. The amount of pure chrtm5 per liter of cell culture is approximately 10 mg.

Liposome preparation. Phospholipids were dissolved in chloroform and dried under a steam of nitrogen. Lipid films were resuspended in reconstitution buffer (25 mM HEPES pH 7.4, 0.1 M KCl). The turbid MLV suspension was passed 21 times through a polycarbonate membrane to give large unilamellar vesicles (LUV) with a diameter of 100 nm. LUVs were solubilized with 20 mM β-OG for one hour at 4° C., and afterwards incubated with protein for an additional hour. The proteoliposome solution was dialyzed with a Liposomat against reconstitution buffer for three hours at room temperature. Pure proteoliposomes were separated from excess protein by gel filtration through a Sephadex G-50 column and were collected by ultracentrifugation at 250000×g for one hour at 4° C. The pellet of pure proteoliposomes was resuspended in reconstitution buffer. The proteoliposomes were used in immunization studies to determine the immunogenicity in rabbits.

### Results

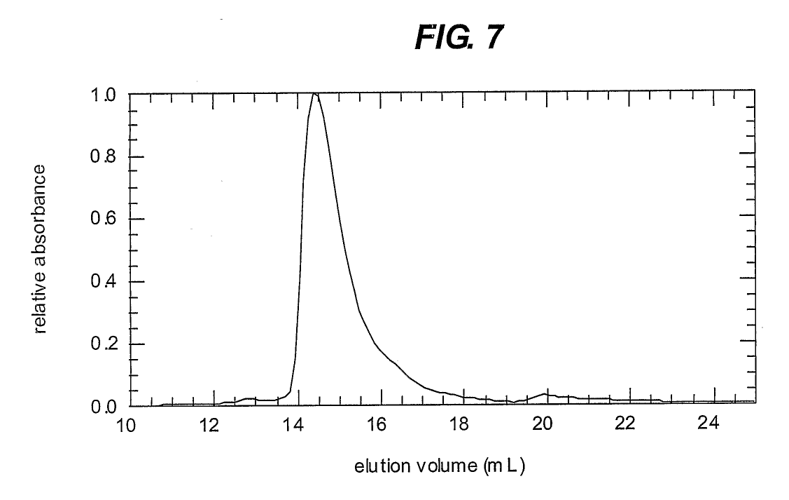

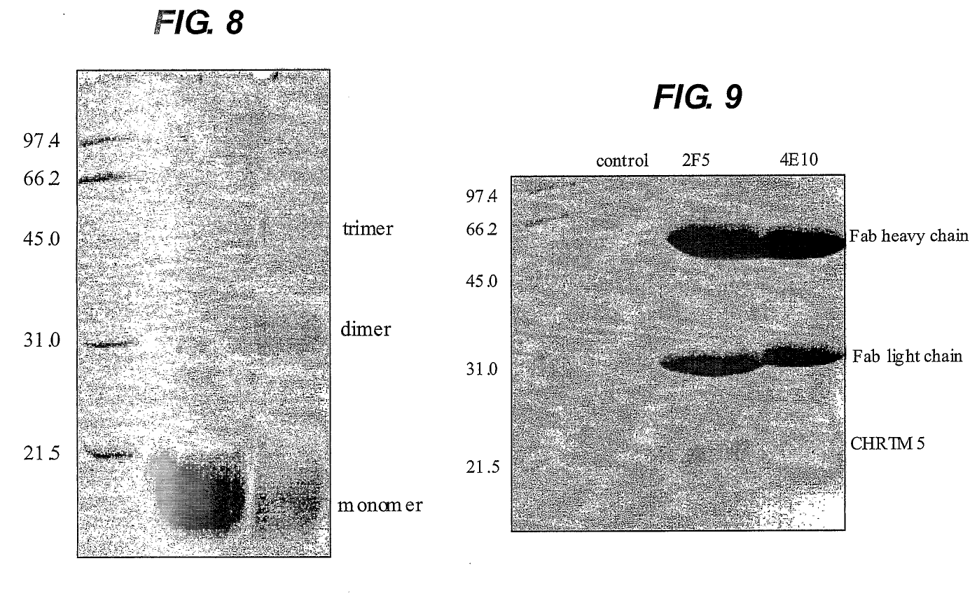

Chrtm5 is overexpressed in E. coli and yields approximately 10 mg per liter culture. The final purification step of chrtm5 on a Superdex 200 gel filtration column in purification buffer (25 mM HEPES pH 7.4, 0.1 M KCl) containing 1% beta octyl-glucopyranoside gives rise to a single peak eluting at 15.8 ml (FIG. 7). This corresponds to a molecular weight that is larger than a single monomer (calculated molecular weight 13.8 kDa) as the marker protein MBP (42 kDa) elutes at a similar position. Chemical cross-linking of chrtm5 suggests that it forms trimers in solution (FIG. 8), which is consistent with the elution profile obtained by gel filtration chromatography.

Pull down assays were then performed to determine whether chrtm5 interacts with the neutralizing antibodies 2F5 and 4E10. This shows that both antibodies pull down chrtm5, while no chrtm5 is pulled down in the control experiment (FIG. 9).

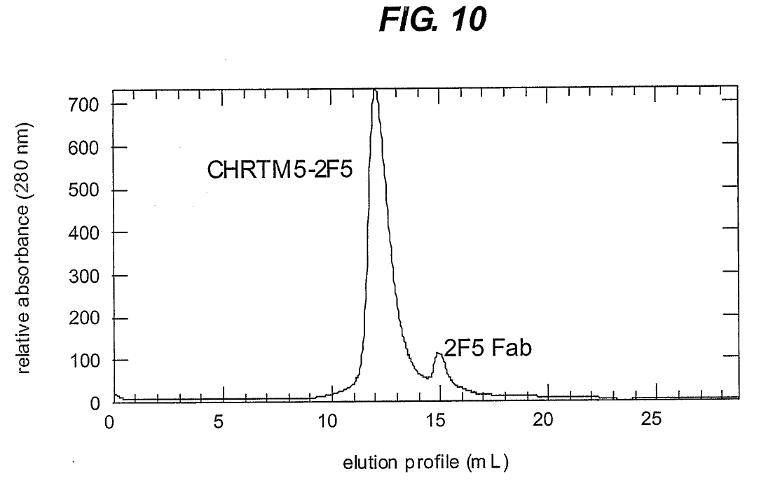

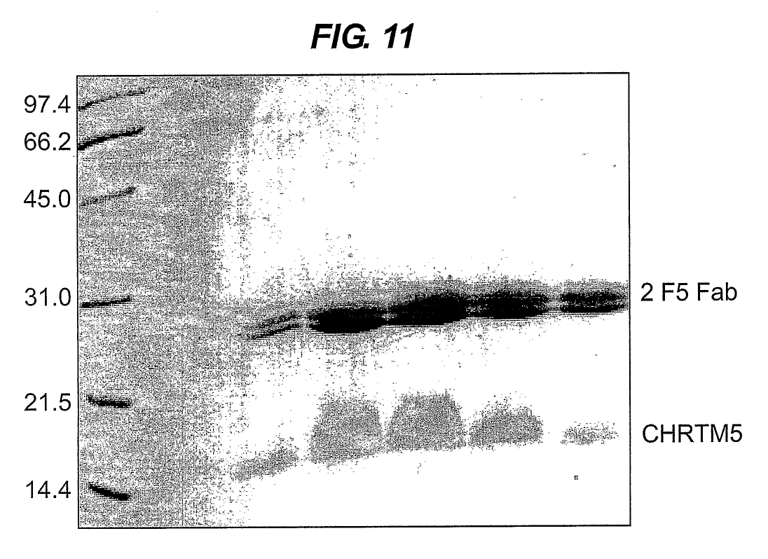

The oligomeric nature of chrtm5 was then further confirmed by purifying chrtm5 in complex with mAb 2F5 Fabs. This complex formation shifts the gel filtration profile to a larger molecular weight and chrtm5/2F5-Fab complexes elute at ˜12.5 ml (FIG. 10). This elution profile is consistent with complex formation as the peak contains both chrtm5 and 2F5 Fab fragments as shown by SDS-PAGE chromatography (FIG. 11). The size of the elution profile suggests further that trimeric gp41 can bind three 2F5 antibodies simultaneously.

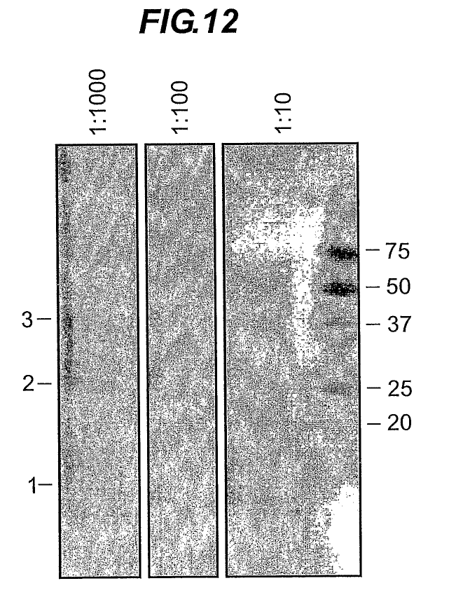

chrtm5 can be incorporated into liposomes, which were used for immunization studies. Preliminary immunization results with the proteoliposomes show that chrtm5 is immunogenic in rabbits after two rounds of immunization. Western blot analysis of one rabbit serum clearly shows that it recognizes the oligomeric forms of chrtm5 (FIG. 12).

It will be understood that the invention has been described by way of example only and modifications may be made whilst remaining within the scope and spirit of the invention.

### REFERENCES

### The Contents of which are Hereby Incorporated by Reference

{1} Root et al (2004) Curr Pharm Design 10:1805-1825
{2} Chen et al (1999) Proc Natl Acad Sci USA 96:8967-8972
{3} Weissenhom et al (1998) Mol Cell 2:605-616
{4} Zhao et al (2000) Proc Natl Acad Sci USA 97:14172-14177
{5} Kobe et al (1999) Proc Natl Acad Sci USA 96:4319-4324
{6} Xu et al (2004) J Biol Chem 279:30514-30522
{7} Tripet et al (2004) J Biol Chem 279:20836-20849
{8} U.S. Pat. No. 5,464,933
{9} U.S. Pat. No. 6,479,055
{10} Bosch et al (2003) J Virol 77:8801-8811
{11} Liu et al (2004) Lancet 363:938-947
{12} Bosch et al (2004) Proc Natl Acad Sci USA 101:8455-8460
{13} Joshi et al (1998) Virology 248:20-34
{14} U.S. Pat. No. 6,750,008
{15} Hildinger et al (2001) J Virol 75:3038-3042
{16} WO 01/37881
{17} Egelhofer et al. 2004 J Virol 78:568-575
{18} Muster et al (1993) J Viro 67:6642-6647
{19} Parker et al (2001) J Virol 75:10906-10911
{20} Stiegler et al (2001) AIDS Res Hum Retroviruses 17:1757-1765
{21} Zwick et al (2004) J Virol 78:3155-3161
{22} Zwick et al (2001) J Virol 75:10892-10905
{23} McGaughey et al (2004) Curr HIV Res 2:193-204
{24} Singh et al (1999) J Mol Biol 290:1031-1041
{25} Gallaher (1996) Cell 85:477-478
{26} Lescar et al (2001) Cell 105:137-148
{27} Chen et al (2002) Protein Sci 11:2774-2791
{28} von Heijne (1992) J Mol Biol 225:487-494
{29} Persson et al (1994) J Mol Biol 237:182-192
{30} Cserzo et al (1997) Protein Engng 10:673-676
{31} Pasquier et al (1999) Protein Engng 12:381-385
{32} Xu et al (2000) Curr Protein Pept Sci 1:1-21
{33} Kukol et al (2002) J Mol Biol 320:1109-1117
{34} Slovic et al (2003) Protein Sci 12:337-348
{35} Harbury et al (1993) Science 262:1401-1407
{36} Weissenhorn et al (1997) Proc Natl Acad Sci USA 94:6065-6069
{37} Weissenhorn et al (1997) Nature 387:426-430
{38} Giannecchini et al (2003) J Virol 77:3724-3733
{39} Wild et al (1994) Proc Natl Acad Sci USA 91:9770-9774
{40} Chan et al (1998) Proc Natl Acad Sci USA 95:15613-15617
{41} U.S. Pat. No. 6,258,782
{42} US Patent Application No. 2004/0044183
{43} Wei et al (2002) Antimicrob Agents Chemother 46:1896-1905
{44} Ibba (1996) Biotechnol Genet Eng Rev 13:197-216
{45} Sambrook (1989) Molecular Cloning; A Laboratory Manual ISBN: 0879695773
{46} Fernandez et al. (1998) eds Gene expression systems. Using nature for the art of expression ISBN: 0122538404
{47} Summers et al. (1987) Texas Agricultural Experiment Station Bulletin No. 1555
{48} U.S. Pat. No. 5,693,506
{49} U.S. Pat. No. 5,659,122
{50} U.S. Pat. No. 5,608,143
{51} Zenk (1991) Phytochemistry 30:3861-3863
{52} Bodanszky (1993) Principles of Peptide Synthesis ISBN: 0387564314
{53} Fields et al (1997) Methods in Enzymology 289: Solid-Phase Peptide Synthesis ISBN: 0121821900
{54} Chan & White (2000) Fmoc Solid Phase Peptide Synthesis ISBN: 0199637245
{55} Kullmann (1987) Enzymatic Peptide Synthesis ISBN: 0849368413
{56} Kazmierski (1999) Peptidomimetics Protocols ISBN: 0896035174
{57} Kirshenbaum et al (1999) Curr Opin Struct Biol 9:530-5
{58} Abell (1999) Advances in Amino Acid Mimetics and Peptidomimetics ISBN: 0762306149
{59} U.S. Pat. No. 5,331,573
{60} Goodman et al (2001) Biopolymers 60:229-245
{61} Hruby & Balse (2000) Curr Med Chem 7:945-970
{62} Ribka & Rich (1998) Curr Opin Chem Biol 2:441-452
{63} Chakraborty et al (2002) Curr Med Chem 9:421-435
{64} Gennaro (2000) Remington: The Science and Practice of Pharmacy 20th ed, ISBN: 0683306472
{65} WO 02/36161
{66} Almeida & Alpar (1996) J Drug Targeting 3:455-467
{67} Pavelic et al (2004) Methods Enzymol 387:287-299
{68} Pavelic et al (2001) Int J Pharm 219:139-149
{69} WO 03/080678
{70} Vaccine design: the subunit and adjuvant approach, eds Powell & Newman, Plenum Press 1995 (ISBN 0-306-44867-X)
{71} Covacci & Rappuoli (2000) J Exp Med 19:587-592
{72} WO 93/18150
{73} Covacci et al (1993) Proc Natl Acad Sci USA 90: 5791-5795
{74} Tummuru et al (1994) Infect Imnun 61:1799-1809
{75} Marchetti et al (1998) Vaccine 16:33-37
{76} Telford et al (1994) J Exp Med 179:1653-1658
{77} Evans et al (1995) Gene 153:123-127
{78} WO 96/01272 & WO 96/01273, especially SEQ ID NO:6
{79} WO 97/25429
{80} WO 98/04702
{81} Gustafsson et al (1996) N Engl J Med 334:349-355
{82} Rappuoli et al (1991) TIBTECH 9:232-238
{83} Vaccines (1988) eds Plotkin & Mortimer ISBN 0-7216-1946-0
{84} Del Guidice et al (1998) Molecular Aspects of Medicine 19:1-70
{85} WO 00/29428
{86} WO 99/27961
{87} WO 02/074244
{88} WO 02/064162
{89} WO 03/028760
{90} Kohler et al (1975) Nature 256:495-497
{91} Kozbor et al (1983) Immunology Today 4:72
{92} Cole et al (1985) Monoclonal Antibodies and Cancer Therapy (Alan R. Liss, Inc.) 77-96
{93} Liu et al (1987) Proc Natl Acad Sci USA 84:3439-3443
{94} Jones et al (1986) Nature 321:522
{95} Verhoeyen et al (1988) Science 239:1534
{96} Kabat et al (1991) J Immunol 147:1709
{97} Queen et al (1989) Proc Natl Acad Sci USA 86:10029
{98} Gorman et al (1991) Proc Natl Acad Sci USA 88:34181
{99} Hodgson et al (1991) Bio/Technology 9:421
{100} McCafferty et al (1990) Nature 348:552-554
{101} Marks et al (1992) Biotechnology 10:779-783
{102} Clackson et al (1991) Nature 352:624-628
{103} Laage et al (2001) Traffic 2:99-104
{104} Laage et al (2000) J Biol Chem 23:17481-17487
{105} Gurezka et al (1999) J Biol Chem 274:9265-9270
{106} Langosch et al (1996) J Mol Biol 263:525-530
{107} Current Protocols in Molecular Biology (F M Ausubel et al, eds. 1987) Supplement 30
{108} Smith & Waterman (1981) Adv Appl Math 2: 482-489
{109} Scianimanico et al (2000) Embo J 19:6732-6741
{110} Wagner et al (2002) J Liposome Res 12:271-283
{111} Wagner et al (2002) J Liposome Res 12:259-270
{112} Reed et al (1938) AM J Hygiene 27:493-497
{113} Eckert et al (2001) Ann Rev Biochem 70:777-810
{114} Center et al (2002) J Virol 76:7863-7867
{115} Chan et al (1998) Proc Natl Acad Sci USA 95:15613-15617
{116} Weissenhom et al. (1999) Mol Membr Biol 16:3-9
{117} Jiang et al (1993) Nature 365:113

1. A fusion inhibitor peptide comprising a sequence derived from an HR2 domain, characterised in that said fusion inhibitor peptide is capable of oligomerization.

2. The peptide according to claim 1, wherein the peptide is capable of trimerization.

3. The peptide according to any one of the preceding claims, wherein the sequence derived from an HR2 domain is linked to a transmembrane domain or a soluble oligomerization domain.

4. The peptide according to claim 3, wherein the transmembrane domain can render the fusion inhibitor peptide capable of oligomerization, preferably trimerization.

5. The peptide according to any one of the preceding claims, wherein said peptide consists of no more than 200 amino acids.

6. The peptide according to any one of the preceding claims, wherein said peptide is of formula NH₂-A-B—C-D-E-COOH, wherein: -A- is an optional N-terminus amino acid sequence consisting of 1 or more amino acids; —B— is a sequence derived from an HR2 domain; —C— is an optional amino acid sequence consisting of 1 or more amino acids; -D- is a transmembrane domain or a soluble oligomerization domain and -E- is an optional C-terminus amino acid sequence consisting of 1 or more amino acids.

7. The peptide according to claim 6, wherein moiety —B— comprises the sequence of T-20, C34 or T-1249, or a functional equivalent thereof.

8. The peptide according to claim 6, wherein the amino acid sequence of moiety —B— is SEQ ID NO: 2, or a functional equivalent thereof.

9. The peptide according to any one of claims 6 to 8, wherein moiety -A- is absent.

10. The peptide according to any one of claims 6 to 9, wherein the sequence of moiety —C— shares at least 50% sequence identity across its length to SEQ ID NO: 4 or SEQ ID NO: 5.

11. The peptide according to any one of claims 6 to 10, wherein moiety -D- is a transmembrane domain that can render the fusion inhibitor peptide capable of oligomerization, preferably trimerization.

12. The peptide according to claim 11, wherein the sequence of moiety -D- is SEQ ID NO: 6, or a functional equivalent thereof.

13. The peptide according to any one of claims 6 to 10, wherein moiety -D- is a soluble oligomerization domain that can render the fusion inhibitor peptide capable of trimerization.

14. The peptide according to claim 13, wherein the sequence of moiety -D- is SEQ ID NO: 7, or a functional equivalent thereof.

15. The peptide according to any one of claims 6 to 14, wherein the sequence of moiety -E- is SEQ ID NO: 8, or a functional equivalent thereof.

16. The peptide according to any one of claims 6 to 16, wherein the sequence of moiety -E- is SEQ ID NO: 22, or a functional equivalent thereof.

17. The peptide according to any one of claims 6 to 14, wherein moiety -E- is absent.

18. The peptide according to any one of claims 6 to 17, wherein: component —B—C— comprises:
i) the monoclonal antibody 2F5 core or extended epitope (SEQ ID NOS: 10 or 11), or a functional equivalent thereof and/or
ii) the monoclonal antibody ZI3 epitope (SEQ ID NO: 12), or a functional equivalent thereof; and/or
iii) the monoclonal antibody 4E10 epitope (SEQ ID NO: 13), or a functional equivalent thereof.

19. The peptide according to any one of claims 1-6, comprising amino acid sequence -A-B—C-D-E-, wherein: -A- is an optional methionine residue; —B— is an amino acid sequence with at least a % sequence identity to SEQ ID NO: 2; —C— is an amino acid sequence with at least b % sequence identity to SEQ ID NOS: 4 or 5; -D- is an amino acid sequence with at least c % sequence identity to SEQ ID NO: 6 or SEQ ID NO: 7; -E- is an optional amino acid sequence with at least d % sequence identity to SEQ ID NO: 8, wherein the value of a, b and c are each independently 50 or more and the value of d is 100.

20. The peptide according to any one of claims 6-15 and 17-19, wherein said peptide comprises SEQ ID NOS: 14, 15, 16 or 17, or a functional equivalent thereof; optionally N-terminally truncated.

21. The peptide according to any one of claims 1-6, comprising amino acid sequence -A-B—C-D-E-, wherein: -A- is an optional amino acid sequence with at least a₁% sequence identity to SEQ ID NO: 20; —B— is an amino acid sequence with at least be % sequence identity to SEQ ID NO: 2; —C— is an amino acid sequence with at least c₁% sequence identity to SEQ ID NO: 4 or SEQ ID NO: 5; -D- is an amino acid sequence with at least d₁% sequence identity to SEQ ID NO: 21 or SEQ ID NO: 7; -E- is an optional amino acid sequence with at least e₁% sequence identity to SEQ ID NO: 22.

22. The peptide according to any one of claims 6-14, 16-18 and 21, wherein said peptide comprises SEQ ID NOS: 23, 24, 25, 26, 27 or 28, or a functional equivalent thereof; optionally N-terminally truncated.

23. A nucleic acid that encodes a peptide of any one of claims 1-22.

24. A vector comprising a nucleic acid of claim 23.

25. A host cell transformed a vector of claim 24.

26. A process for producing a peptide of the invention, comprising the step of culturing a host cell of claim 25 under conditions that induce expression of the peptide.

27. A peptidomimetic compound of a peptide according to any one of claims 1 to 22, wherein the peptidomimetic compound is a fusion inhibitor.

28. A pharmaceutical composition comprising (a) according to any one of claims 1 to 22 and (b) a pharmaceutical carrier.

29. The pharmaceutical composition of claim 28, wherein the pharmaceutical carrier is a liposome.

30. The pharmaceutical composition of claim 29, wherein the peptide is incorporated in the liposome by means of a transmembrane domain in the peptide.

31. The pharmaceutical composition of claim 29 or 30, wherein the peptide is incorporated in the liposome such that the sequence derived from an HR2 domain is on the outside face of the liposome.

32. The pharmaceutical composition of any one of claims 29 to 31, wherein pharmaceutical composition is prepared as a liposomal gel for topical administration.

33. The pharmaceutical composition of any one of claims 28 to 32, wherein the composition is an immunogenic composition.

34. The peptide according to any one of claims 1 to 22, for use as a medicament.

35. A method for treating a subject suffering from or at risk of contracting an infection, comprising administering to the subject a pharmaceutical composition according to any one of claims 28 to 33.

36. Use of a pharmaceutical composition according to any one of claims 28 to 33 in the manufacture of a medicament for treating a subject.

37. A method for expressing a protein comprising one or more transmembrane domain(s) in an expression system, said method comprising the steps of:
a) fusing a sequence encoding an 18-mer peptide of SEQ ID NO: 8 or a functional equivalent thereof to a gene encoding said protein; and
b) expressing the resultant gene-fusion product in an expression system.

38. The method according to claim 37, wherein the expression system is a prokaryotic expression system.

39. The method according to any one of claims 37 or 38, the expression system is an E. coli expression system.

40. A nucleic acid that encodes an 18-mer peptide of SEQ ID NO: 8 or a functional equivalent thereof.

41. A vector comprising a nucleic acid according to claim 40.

42. The vector according to claim 41, wherein said nucleic acid is fused to a gene encoding a protein comprising one or more transmembrane domain(s).

43. The method according to any one of claims 37 to 39, or the vector according to claim 42, wherein at least one sequence encoding a transmembrane domain is located at the 3′ or 5′ end of the gene encoding the protein comprising one or more transmembrane domain(s).

44. The method according to any one of claims 37 to 39 or 43, or the vector according to any one of claims 42 to 43, wherein the sequence encoding the 18-mer peptide or a functional equivalent thereof is fused to the 3′ or 5′ end of the gene encoding the protein comprising one or more transmembrane domain(s).

45. The method according to any one of claims 37 to 39 or 43 to 44, or the vector according to any one of claims 42 to 44, wherein the gene encoding the protein comprising a transmembrane domain is a heterologous gene.

46. The method according to any one of claims 37 to 39 or 43 to 45, or the vector according to any one of claims 42 to 45, wherein the gene encoding the protein comprising a transmembrane domain is an eukaryotic gene.


SEQUENCE LISTING (28 sequences)

SEQ ID NO: 1 - protein, 345 aa, Artificial
  Synthetic peptide
AVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 2 - protein, 37 aa, Artificial Sequence
  Synthetic peptide
MEWDREINNYTSLIHSLIEESQNQQEKNEQELLELDK

SEQ ID NO: 3 - protein, 117 aa, Artificial Sequence
  Synthetic peptide
AVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTW

SEQ ID NO: 4 - protein, 18 aa, Artificial Sequence
  Synthetic peptide
WASLWNWFNITNWLWYIK

SEQ ID NO: 5 - protein, 18 aa, Artificial Sequence
  Synthetic peptide
WASLWNWFDITNWLWYIK

SEQ ID NO: 6 - protein, 23 aa, Artificial Sequence
  Synthetic peptide
LFIMIVGGLVGLRIVFAVLSIVN

SEQ ID NO: 7 - protein, 31 aa, Artificial Sequence
  Synthetic peptide
KQIEDKIEEILSKIYHIENEIARIKKLIGEA

SEQ ID NO: 8 - protein, 18 aa, Artificial Sequence
  Synthetic peptide
APRALEIILFNFKKEIYI

SEQ ID NO: 9 - protein, 150 aa, Artificial Sequence
  Synthetic peptide
RVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 10 - protein, 6 aa, Artificial Sequence
  Synthetic peptide
ELDKWA

SEQ ID NO: 11 - protein, 16 aa, Artificial Sequence
  Synthetic peptide
NEQELLELDKWASLWN

SEQ ID NO: 12 - protein, 21 aa, Artificial Sequence
  Synthetic peptide
LLELDKWASLWNWFDITNWLW

SEQ ID NO: 13 - protein, 6 aa, Artificial Sequence
  Synthetic peptide
NWFNIT

SEQ ID NO: 14 - protein, 96 aa, Artificial Sequence
  Synthetic peptide
MEWDREINNYTSLIHSLIEESQNQQEKNEQELLELDKWASLWNWFNITNWLWYIKLFIMIVGGLVGLRIVFAVLSIVNAPRALEIILFNFKKEIYI

SEQ ID NO: 15 - protein, 96 aa, Artificial Sequence
  Synthetic peptide
MEWDREINNYTSLIHSLIEESQNQQEKNEQELLELDKWASLWNWFDITNWLWYIKLFIMIVGGLVGLRIVFAVLSIVNAPRALEIILFNFKKEIYI

SEQ ID NO: 16 - protein, 78 aa, Artificial Sequence
  Synthetic peptide
MEWDREINNYTSLIHSLIEESQNQQEKNEQELLELDKWASLWNWFNITNWLWYIKLFIMIVGGLVGLRIVFAVLSIVN

SEQ ID NO: 17 - protein, 78 aa, Artificial Sequence
  Synthetic peptide
MEWDREINNYTSLIHSLIEESQNQQEKNEQELLELDKWASLWNWFDITNWLWYIKLFIMIVGGLVGLRIVFAVLSIVN

SEQ ID NO: 18 - protein, 60 aa, Artificial sequence
  Synthetic peptide
EKNEQELLELDKWASLWNWFNITNWLWYIKQIEDKIEEILSKIYHIENEIARIKKLIGEA

SEQ ID NO: 19 - protein, 133 aa, Artificial sequence
  Synthetic peptide
MHHHHHHKQIEDKIEEILSKIYHIENEIARIKKLIGEAEWDREINNYTSLIHSLIEESQNQQEKNEQELLELDKWASLWNWFNITNWLWYIKLFIMIVGGLVGLRIVFAVLSIVNAPRALEIILFNFKKEIYI

SEQ ID NO: 20 - protein, 32 aa, Artificial sequence
  Synthetic peptide
MKHHHHHHPMSDYDIPTTENLYFQGAMDDDDK

SEQ ID NO: 21 - protein, 23 aa, Artificial sequence
  Synthetic peptide
LFIMIVGGLVGLRIVFAVLSVVN

SEQ ID NO: 22 - protein, 5 aa, Artificial sequence
  Synthetic peptide
RVRQG

SEQ ID NO: 23 - protein, 115 aa, Artificial sequence
  Synthetic peptide
MKHHHHHHPMSDYDIPTTENLYFQGAMDDDDKMEWDREINNYTSLIHSLIEESQNQQEKNEQELLELDKWASLWNWFNITNWLWYIKLFIMIVGGLVGLRIVFAVLSVVNRVRQG

SEQ ID NO: 24 - protein, 115 aa, Artificial sequence
  Synthetic peptide
MKHHHHHHPMSDYDIPTTENLYFQGAMDDDDKMEWDREINNYTSLIHSLIEESQNQQEKNEQELLELDKWASLWNWFDITNWLWYIKLFIMIVGGLVGLRIVFAVLSVVNRVRQG

SEQ ID NO: 25 - protein, 110 aa, Artificial sequence
  Synthetic peptide
MKHHHHHHPMSDYDIPTTENLYFQGAMDDDDKMEWDREINNYTSLIHSLIEESQNQQEKNEQELLELDKWASLWNWFNITNWLWYIKLFIMIVGGLVGLRIVFAVLSVVN

SEQ ID NO: 26 - protein, 110 aa, Artificial sequence
  Synthetic peptide
MKHHHHHHPMSDYDIPTTENLYFQGAMDDDDKMEWDREINNYTSLIHSLIEESQNQQEKNEQELLELDKWASLWNWFDITNWLWYIKLFIMIVGGLVGLRIVFAVLSVVN

SEQ ID NO: 27 - protein, 83 aa, Artificial sequence
  Synthetic peptide
MEWDREINNYTSLIHSLIEESQNQQEKNEQELLELDKWASLWNWFNITNWLWYIKLFIMIVGGLVGLRIVFAVLSVVNRVRQG

SEQ ID NO: 28 - protein, 83 aa, Artificial sequence
  Synthetic peptide
MEWDREINNYTSLIHSLIEESQNQQEKNEQELLELDKWASLWNWFDITNWLWYIKLFIMIVGGLVGLRIVFAVLSVVNRVRQG